SEQUENCE LISTING (35 sequences)

SEQ ID NO: 1 - protein, 9 aa, Artificial sequence
  Synthetic peptide
SLSRLSGTV

SEQ ID NO: 2 - protein, 9 aa, Mus musculus
MCLRMTAVM

SEQ ID NO: 3 - protein, 9 aa, Homo sapiens
LLSAEPVPA

SEQ ID NO: 4 - protein, 9 aa, Homo sapiens
LLGPRLVLA

SEQ ID NO: 5 - protein, 9 aa, Homo sapiens
SLWGQPAEA

SEQ ID NO: 6 - protein, 9 aa, Homo sapiens
VLAPRVLRA

SEQ ID NO: 7 - protein, 9 aa, Homo sapiens
ALVVQVAEA

SEQ ID NO: 8 - protein, 8 aa, Homo sapiens
HGVFLPLV

SEQ ID NO: 9 - protein, 9 aa, Homo sapiens
LLAAWTARA

SEQ ID NO: 10 - protein, 10 aa, Homo sapiens
VLLKARLVPA

SEQ ID NO: 11 - protein, 11 aa, Homo sapiens
KMDASLGNLFA

SEQ ID NO: 12 - protein, 11 aa, Homo sapiens
LLFSHVDHVIA

SEQ ID NO: 13 - protein, 9 aa, Homo sapiens
FLGPWPAAS

SEQ ID NO: 14 - protein, 9 aa, Homo sapiens
SLYALHVKA

SEQ ID NO: 15 - protein, 10 aa, Homo sapiens
LLLSAEPVPA

SEQ ID NO: 16 - protein, 10 aa, Homo sapiens
AMAPPSHLLL

SEQ ID NO: 17 - protein, 9 aa, Homo sapiens
MAPLALHLL

SEQ ID NO: 18 - protein, 10 aa, Homo sapiens
FLLGPRLVLA

SEQ ID NO: 19 - protein, 10 aa, Homo sapiens
LLLDVPTAAV

SEQ ID NO: 20 - protein, 12 aa, Homo sapiens
LLLDVPTAAVQA

SEQ ID NO: 21 - protein, 9 aa, Homo sapiens
LLLDVPTAA

SEQ ID NO: 22 - protein, 9 aa, Homo sapiens
LLDVPTAAV

SEQ ID NO: 23 - protein, 14 aa, Homo sapiens
VLFRGGPRGLLAVA

SEQ ID NO: 24 - protein, 9 aa, Homo sapiens
ALLSSLNDF

SEQ ID NO: 25 - protein, 9 aa, Homo sapiens
IITKEVLAP

SEQ ID NO: 26 - protein, 11 aa, Homo sapiens
QLQEGKNVIGL

SEQ ID NO: 27 - protein, 11 aa, Homo sapiens
ILAPAGSLPKI

SEQ ID NO: 28 - protein, 10 aa, Homo sapiens
MASRWGPLIG

SEQ ID NO: 29 - protein, 12 aa, Homo sapiens
AVLALVLAPAGA

SEQ ID NO: 30 - protein, 8 aa, Homo sapiens
LAPRVLRA

SEQ ID NO: 31 - protein, 10 aa, Homo sapiens
AALLDVRSVP

SEQ ID NO: 32 - protein, 9 aa, Homo sapiens
SLPKKLALL

SEQ ID NO: 33 - protein, 9 aa, Homo sapiens
KAPVTKVAA

SEQ ID NO: 34 - protein, 9 aa, Homo sapiens
AMAQLLLVL

SEQ ID NO: 35 - protein, 9 aa, Homo sapiens
NPLPSKETI

(19) United States
(12) Patent Application Publication
(10) Pub. No.: US 2009/0220534 A1
(43) Pub. Date: Sep. 3, 2009

(54) METHODS FOR IDENTIFYING T-CELL EPITOPES ASSOCIATED WITH IMPAIRED PEPTIDE PROCESSING AND APPLICATIONS OF THE IDENTIFIED EPITOPES

(75) Inventors: Thorbald van Hall, Alphen a/d Rijn (NL); Andreas Oliver Weinzierl, Tübingen (DE); Petrus Antonius van Veelen, Wassenaar (NL); Jan Wouter Drijfhout, Leiden (NL); Cornelis Johannes Maria Melief, Haarlem (NL); Rienk Offringa, Leiden (NL)

Correspondence Address: FOLEY AND LARDNER LLP;SUITE 500, 3000 K STREET NW, WASHINGTON, DC, 20007, US

(73) Assignee: Leiden University Medical Center

(21) Appl. No.: 12/293,054

(22) PCT Filed: Mar. 16, 2007
(86) PCT No.: PCT/NL2007/050105

§ 371 Date: Feb. 12, 2009

(30) Foreign Application Priority Data
Mar. 16, 2006 (EP) 06111248.8

(51) Int. Cl. A61K 39/00; C12N 5/10; C12N 15/63; C40B 30/04; C07K 7/08; C07H 21/04

(52) U.S. Cl. 424/185.1; 435/325; 435/320.1; 506/9; 530/326; 530/327; 536/23.1

(57) ABSTRACT

The current invention provides methods for the identification of antigens and/or epitopes that are differentially displayed on TAP deficient or TAP impaired cells and are not detectably displayed on normal or TAP proficient cells. The identification and applications of these differentially presented antigens, which in this specification are referred to as TEIPP, T cell Epitopes associated with Impaired Peptide Processing, is a prime object of this invention. The invention also provides peptides comprising a TEIPP epitope obtained from the methods of the invention, which may be applied in medicaments and methods of treatment raising a T cell response against TAP deficient tumor cells or virally infected cells.

### FIELD OF THE INVENTION

The current invention relates to the fields of medicine, in particular to the fields of immunology, vaccination and treatment of viral infections and cancer.

### BACKGROUND OF THE INVENTION

CD8+ cytotoxic T lymphocytes (CTL) play an important role in the immune defense against viral infections and have also shown to be highly effective in controlling tumor growth (1, 2). The inhibition of MHC class I-restricted antigen presentation is therefore an attractive strategy for viruses and cancers to evade immune-mediated destruction (3, 4). A defect frequently observed in virus-infected cells and in tumors constitutes impairment of the transporter associated with antigen processing (TAP) (5-7). It is a heterodimeric protein, made up of subunits TAP1 and TAP2, that is localized in the Endoplasmic Reticulum (ER). TAP is a member of the ATP-binding cassette (ABC) transporter family of proteins. These proteins require the binding of ATP in order to drive the transport of molecules. Each TAP subunit has an N-terminal membrane-spanning domain and a C-terminal ABC ATPase domain. The transporter (TAP1:TAP2) is responsible for the delivery into the endoplasmic reticulum (ER) of the cytosolic peptide repertoire resulting from proteolytic breakdown of intracellular proteins, thereby making it available for loading onto MHC class I molecules (8). It functions during the loading of peptides onto partially folded Major Histocompatability Complex Class I (MHC I). Peptides comprised of 8 to 16 amino acids bind to TAP with similar affinity. However, peptide translocation occurs most often for peptides 8 to 12 amino acids in length. Experiments have shown peptides of up to 40 amino acids to translocate through the TAP complex. It appears that TAP binds peptides that are of optimal length, or slightly larger than those presented by MHC I. MHC I molecules generally load peptides of 8 to 10 amino acids long.

The importance of TAP function for the display of peptide epitopes by MHC class I is underscored by the greatly diminished expression of MHC class I molecules at the surface of TAP-deficient cells (9). This indicates that the vast majority of class I molecules at the surface of processing-proficient cells comprise peptide epitopes of which the delivery to the ER depends on TAP. Accordingly, suppression or loss of TAP-function generally results in failure of target cell recognition by epitope-specific effector CTL (10-12). In the search for immune effector mechanisms that could counteract the escape of TAP-deficient target cells from CTL-mediated destruction, a category of T-cells was uncovered that is capable of eliminating cells with defects in MHC class I antigen processing. The inventors initial finding was that immunization of mice with B7.1-expressing, TAP-deficient RMA-S lymphoma cells elicited a novel type of T cell response (13). These CD8+ T cells selectively killed TAP-deficient cells, suggesting the existence of a new CTL target structure.

EP 0964 697 is directed at some practical applications of the observation that T cells can be activated against epitopes on cells that are TAP deficient. TAP deficient cells are frequently tumour cells or virally infected cells. EP 0964 697 teaches the use of compounds that inhibit TAP function, to impair peptide processing for MHC presentation, thereby making these cells vulnerable for T-cells specific for these unidentified antigens on TAP deficient cells. The description teaches that antigens and epitopes associated with impaired TAP function could potentially be used for immunization against cancer or as a virus vaccine. However, EP 0964 697 does not disclose the nature of the antigens or epitopes on TAP deficient cells, nor does it disclose a method of identifying these antigens or epitopes.

In a previous publication (13) the inventors hypothesized that T-cells can be directed against a repertoire of peptides that normally are not presented at significant levels by MHC class I molecules in TAP proficient cells, but that are presented by MHC class I molecules on TAP deficient cells. Alternatively, these CTL's were reported to potentially recognize epitopes and antigens that are not of peptidic nature or recognize epitopes that are formed of the MHC class I molecules themselves. The authors concluded that the structure of MHC class I molecules on TAP deficient cells is not known (13).

Hence, the nature of MHC class I antigens and epitopes displayed on TAP deficient or impaired cells was hitherto unidentified. The identification of antigens and epitopes on TAP deficient cells, in particular tumour cells or virally infected cells, has been postulated to provide excellent targets for vaccination strategies against tumour cells or against viruses. The current invention provides methods for the identification of MHC class I antigens and epitopes that are differentially displayed on TAP deficient or TAP impaired cells and are not detectably displayed on normal or TAP proficient cells. Such epitopes are herein referred to as TEIPP, T cell Epitopes associated with Impaired Peptide Processing. The identification and applications of these differentially presented antigens, comprising TEIPP, is a prime object of this invention.

### SUMMARY OF THE INVENTION

The current invention addresses the problem of identifying TEIPP by making a detailed analysis of the MHC-restriction and antigen identity of T cells specific for TAP impaired or TAP deficient cells. Data reveal the existence of a previously unknown peptide repertoire that is presented at the surface of processing-deficient cells in the context of both classical and non-classical MHC class I molecules. In view of the importance and potential of these T cells in targeting viral infections or tumours that are associated with impaired TAP-function, their efficacy in eradicating TAP-deficient cells in vivo was determined. The inventors established that induction of this CTL response, either by adoptive transfer of ex vivo expanded CTLs or by immunization with synthetic peptide epitopes, can result in the control of TAP-deficient tumours in vivo, illustrating a practical application and the potential of the current invention.

The current invention hence provides methods for identifying MHC class I binding peptides comprising TEIPP epitopes, the method comprising:

a) providing a cell deficient in TAP transport, AND
b1) inducing and isolating a TEIPP specific T-cell against the TAP deficient cells in a mammal,
b2) screening of a peptide library with an isolated TEIPP specific T-cell and identification of one or more mimotope sequences that are recognized by the T-cell; and
b3) determination of the T cell-specific recognition motif by substituting single amino acids in a mimotope sequences identified in (b2); and
b4) screening one or more databases of naturally occurring polypeptide sequences with the recognition motif to identify a naturally occurring peptide that matches the motif; OR,
c) performing mass-spectrometric analysis of purified natural peptides displayed on MHC molecules obtained from TAP deficient cells.

The invention also provides peptides comprising an epitope obtained from the method of the invention, which may be suitably applied in medicaments and methods of treatment to raise a T cell response against tumor cells or virally infected cells.

### DETAILED DESCRIPTION OF THE INVENTION

Deficiencies in MHC class I antigen presentation are known to provide target cells an opportunity to escape CTL-mediated immune defense. Therefore, the discovery of TEIPP-specific CTL's, which display a clear preference for target cells with impairments in antigen processing and very low surface levels of MHC class I, would be highly advantageous for the development of therapies and medicaments for the treatment of infectious diseases and cancer.

The methods according to this invention allow for the identification of peptides comprising TEIPP epitopes. The invention sheds light on the molecular identity of TEIPP epitopes and provides compelling insights into a novel repertoire of immunogenic epitopes that make it to the surface of processing deficient cells. Even though these epitopes are derived from commonly expressed self proteins, the immune system considers them as neo antigens, due to their absence from the surface of normal, TAP-proficient cells.

Identification of the target epitopes for the available TEIPP-specific CTL clones is hampered by the very low amounts of MHC class I molecules that can be isolated from the cell surface of processing-deficient, TAP^{-/-} cells, in combination with the considerable diversity of peptides contained within these isolates. These technical difficulties are overcome according to the teachings of this invention by functional screening of synthetic peptide libraries and by combining the outcome of such screenings with those of database searches and optionally the mass spectrometric analysis of eluted natural epitopes. A striking aspect of this procedure is that the mimotopes sequences initially isolated from the peptide library can diverge significantly from the peptide recognized by the TEIPP specific CTL clone in vivo. This is for instance illustrated in the examples, where a TEIPP mimotope identified (SLSRLSGTV) has only one position in common with the natural TEIPP epitope sequence (MCLRMTAVM) identified. This does not only illustrate the high degree of flexibility in TCR-mediated peptide recognition, but also reflects the crucial importance of integrating the results from library screenings with data on eluted natural epitopes, as taught by this specification. The multi-disciplinary approach according to this invention can be applied for the identification of any TEIPP epitope and sequence.

In a first embodiment the invention provides a method for identifying a MHC class I binding peptide comprising a TEIPP epitope, whereby the method comprises the steps of:

a) providing a cell deficient in TAP transport, AND
b1) inducing and isolating a TEIPP specific T-cell against the TAP deficient cells in a mammal,
b2) screening of a peptide library with an isolated TEIPP specific T-cell and identification of one or more mimotope sequences that are recognized by the T-cell; and
b3) determination of the T cell-specific recognition motif by substituting single amino acids in a mimotope sequences identified in (b2); and
b4) screening one or more databases of naturally occurring polypeptide sequences with the recognition motif to identify a naturally occurring peptide that matches the motif; OR,
c) performing mass-spectrometric analysis of purified natural peptides displayed on MHC molecules obtained from TAP deficient cells.

The peptide libraries may be of biological origin or more preferably synthetically made, and are of a complexity of at least 100,000 different peptides, more preferably 500,000 or more. Individual peptides are 8 to 16, more preferably between about 8 and 12 amino acids in length, most preferably 8-9 amino acids, although longer peptides or fragments may be used. An example of a library of peptides from biological origin is provided in example 8. A mimotope is defined as a stimulating peptide ligand.

Peptide libraries can consist of sets of individually obtained peptides, preferably obtained by chemical synthesis. Although equipment for synthesis of a multitude of individual peptides is commercially available, the synthesis of hundreds of thousands of individual peptides is very costly and time-consuming. In a preferred embodiment peptide libraries of the complexity required are obtained by solid phase peptide synthesis via a mix and split, one-bead-one-peptide approach as exemplified in this specification.

In contrast to the identification of antibody mimotopes that can be performed with solid carrier-bound peptides, the identification of T-cell mimotopes requires the application of peptides in solution. In a preferred embodiment one-bead-one-peptide libraries are constructed in such a way that screening of peptide activity can be performed with peptides in solution, whereas selection and identification of peptides can be performed with bead-bound peptides. For this purpose hybrid beads in combination with controlled release of peptides may be applied. On a hybrid bead part of the peptide molecules is attached to the bead by an acid-stabile linkage (acid-stable attached peptide, ASA-P) whereas the other part of the peptide molecules is attached in acid-labile form (acid-labile attached peptide, ALA-P). Furthermore, the ASA-P is attached to the bead in such a way that ASA-P can be removed from the beads in portions. This enables the removal of part of the ALA-P from the same bead at different stages of the converging screening, whereas the ASA-P is still available for identification by Edman sequencing of the bead of interest. As can be seen from the examples the above invention could be performed using hybrid resins containing about 25% ASA-P and 75% ALA-P in which a protocol was used with which the ALA-P was removed in three steps. Hybrid beads may be generated by coupling a mixture of Fmoc-Nle-OH and the 3-(4-hydroxymethylphenoxy)propionic acid ester of Fmoc-Val-OH to a TentagelS amine resin. Alternatively, TEIPP specific T-cell clones can be used to screen peptide libraries using any of the commonly known techniques available to the skilled artisan.

The databases to be used for identifying matching epitopes in naturally occurring sequences may be any proprietary or public database such as NCBI GenBank, EMBL, SwissProt and the like.

TEIPP epitopes may also be isolated by differential screening of cells proficient in TAP transport and closely related cells deficient in TAP transport. This may be achieved for instance by inactivation of TAP function using viruses or viral proteins, or by genetic modification of cells, TAP^{-/-} and TAP^{+/+} cells as described in the examples section, wherein the gene encoding TAP1 and/or TAP2 is inactivated or TAP function is inactivated by other means, such as for instance viral antigens. More preferably, TAP function in TAP deficient cells may be restored by providing cells with TAP1 and/or TAP2 expression, to provide cell lines which differ in TAP function but are otherwise identical. MHC bound epitopes can be eluted from TAP^{-/-} and TAP^{+/+} and differentially displayed peptides may be purified and analysed using sequencing and/or mass spectrometry techniques and differential (isotope) labelling techniques known in the art (Lemmel et al. Nature Biotechnology 22:450, 2004). The putative TEIPP epitopes thus identified may either be used as an input for screening of potential or established CD8+ TEIPP specific T-cells, or for optimization purposes and by substituting amino acids in these TEIPP specific epitopes. Alternatively such putative TEIPP epitopes may be used to generate CD8+ T-cell responses in vitro or in vivo by peptide immunizations, which are well known and documented in the art, for instance in WO 02 070006.

In another embodiment the invention provides peptides comprising a TEIPP epitope, obtained from the methods described above. Such a TEIPP epitope comprising peptide may be extended or trimmed to have a preferred length between 22 and 45 amino acids. Although the epitope itself is normally between 8 and 16, preferably between 8 and 12 and mostly 8 or 9 amino acids, the peptide is preferably elongated with additional amino acids. The TEIPP epitope may be flanked with additional amino acids on either the N-terminal or C-terminal end of the epitope, or on both ends. Within a peptide according to the invention, the TEIPP epitope is preferably flanked by at least one of processing-dependent sequences, T helper and CTL epitopes.

Processing-dependent amino acid sequences for flanking of TEIPP epitopes in the peptides of the invention are herein understood to mean amino acid sequence that are processed away from natural peptides comprising T cell epitopes (other than the TEIPP epitope) in cells that are proficient in pathways for processing of peptide into MHC class I presented peptides, i.e. epitopes. Preferably, the processing-dependent amino acid sequences are sequences that are processed by the ‘proteasome-TAP processing pathway’ in TAP proficient cells. The majority of MHC class I presented peptides results from liberation by the proteasome enzyme and from transport to the ER by TAP. TEIPP peptides are, however, presented in MHC class I molecules via TAP- and proteasome-independent mechanism. In a preferred embodiment of the invention, a peptide of the invention comprising a TEIPP epitope is elongated on either the N-terminal or C-terminal end of the epitope, or on both ends of at least one minimal TEIPP-epitopes with processing-dependent flanking amino acid sequences as defined above, i.e. that are derived from regions naturally flanking known epitopes. Regions naturally flanking known epitopes may be derived from a variety of antigens, including tumor antigens, viral antigens and the like. The goal of using such processing dependent flanking sequences is to route the peptides of the invention comprising TEIPP epitopes through this pathway the classical ‘proteasome-TAP’ machinery in TAP proficient cells s as to more efficiently induce a CTL response to these epitopes as presented on processing deficient tumor or virally infected cells. Alternatively, natural amino acid flanking TEIPP epitopes might be used.

Thus preferred the peptides of the invention have length in excess of 10, 11, 12, 13, 14, 15 or 16 amino acids. The size is chosen such that direct fit/binding into the groove of an MHC molecule is prevented and that the peptide must be processed before being capable of presentation in the context of an MHC molecule. At the same time the polypeptides of the invention may be not naturally occurring proteins in which the epitopes are present and are preferably of synthetic origin. Chemical synthesis of a peptide puts a practical limit on the length of the peptide. Therefore the length of the peptides of the invention is preferably less than 120, 100, 80, 60, 50 or 45 amino acids. These and other advantageous effects of the preferred length of e.g., 14-80 or 16-60, more preferably 20-50, most preferably 22-45 amino acids of the peptides for diagnostic- and immunization purposes can for instance be found in WO 02/070006. In the polypeptides of the invention the amino acids in excess of 10, 11, 12, 13, 14, 15 or 16 amino acids are not necessarily contiguous amino acids from the antigen that comprise the T-cell epitope, the excess amino acid may be heterologous to the antigen or even to the species from which the antigen is derived. A total length of the TEIPP epitope as just described is highly advantageous for raising T cell responses by presentation and activation via professional antigen presenting cells, as is described in WO 02 070006.

A TEIPP epitope comprising peptide according to the invention may advantageously further comprise a T helper epitope. The presence of a T-helper epitope, which can be presented by MHC class II molecules on professional antigen presenting cells, comprised in the peptide of the invention is preferred because T-helper cells will upregulate CD40 ligand expression and thereby activate professional antigen presenting cells (APC), such as dendritic cells, via CD40 activation. Activation of dendritic cells provide positive T cells, preferably CTLs, specific for the TEIPP epitope with a “license to kill”. Preferably the TEIPP epitopes are located in the C-terminus of the peptide and the T-helper epitope more central or in the N-terminus of the peptide.

A TEIPP epitope comprising peptide is preferably presented on the surface of a cell on an MHC class I molecule, preferably but not exclusively on TAP deficient or TAP impaired cells, such as, but not limited to: virally infected cells, tumor cells and otherwise immortalized and/or transformed cells. The TEIPP epitope comprising peptides may be presented on ‘classical’ MHC class I molecules such as murine K^{b} or D^{b} and human HLA-A, HLA-B, HLA-C, but also on non-classical MHC molecules such as murine Qa-1^{b} and M3, or human HLA-E, HLA-F, HLA-G or HLA-H.

Most preferably the peptide according to the invention comprises a sequence selected from the group of epitopes consisting of SEQ ID No's 1 to SEQ ID No. 35 in this specification.

The ER of mammalian (and human) cells contains MHC class I-binding peptides that can reach the ER compartment independently of proteasome degradation and TAP-function/transport. In TAP proficient cells such peptides may fail to become loaded into MHC class I in sufficient levels due to competition by the overwhelming amounts of TAP-dependent peptides. Indeed, human TAP-deficient cells are shown here to present a unique set of TAP-independent peptides in their surface MHC class I molecules. A fraction of the TEIPP repertoire comprises of membrane spanning proteins, in particular fragments which normally reside on the luminal side of the ER membrane. Another significant fraction of the TEIPP repertoire that was discovered by this invention, are signal peptide sequences. A signal peptide is a short (15-60 amino acids long) peptide chain that directs the post translational transport of a protein. Some signal peptides are cleaved from the protein by signal peptidase after the proteins are transported. Signal peptides may also be called targeting signals or signal sequences. The amino acid sequences of signal peptides direct proteins which are synthesized in the cytosol to certain organelles such as the nucleus, mitochondrial matrix, endoplasmic reticulum, chloroplast, and peroxisome. As demonstrated in the examples section, the major part of the identified human TEIPP epitopes differentially expressed on TAP deficient cells are derived from signal sequences, in particular those that are cleaved off upon transfer of the peptide through the membrane. The invention thus provides for the use of a signal peptides derived from and/or cleaved off upon transport of a protein over a membrane, for the manufacture of a medicament for induction of an immune response against TAP deficient cells. This use will be particularly advantageous for medicaments and methods of treatment aimed at the induction of an immune response against a virally infected or tumor cells deficient in TAP transport.

Furthermore, most of the identified TEIPP epitope comprising peptides comply with the C-end rule. Hence, in a preferred embodiment, the invention provides the use of signal peptides or fragments thereof, preferably complying with the C-end rule, as TEIPP epitopes or putative TEIPP epitopes, for which a CD8+ T cell response may be actively raised or identified among T cell populations, or may be used as a starting point for optimization in the screening method of this invention.

In another embodiment the invention provides nucleic acid sequences and nucleic acid molecules encoding the TEIPP epitope comprising peptide according to the invention. Preferably, the nucleic acid sequences according to this invention are comprised in a nucleic acid vector capable of conferring expression of TEIPP epitope peptides or proteins in a host cell. The nucleic acid vector may be any DNA or RNA vector known in the art, integrating or non-integrating, such as but not limited to: a plasmid, cosmid, episome, artificial chromosome, (retro-)virus or phage. The invention also pertains to a host cell comprising the nucleic acid comprising a TEIPP epitope coding sequence or a vector according to this invention. The host cell may be any prokaryote or eukaryote cell, and preferably is a mammalian or human cell, expressing a coding sequence comprising the TEIPP epitope. In a preferred embodiment a peptide comprising the TEIPP epitope is secreted and/or displayed on the surface of the cell.

A TEIPP comprising peptide may be synthetically prepared via standard procedures or may be prepared by biological processes, such as via recombinant DNA technology and expression in host cells, such as mammalian cells, bacterial cells, insect cells or yeast cells. Recombinant expression technology is well known in the art and may for instance be found in: Molecular Cloning, Maniatis Sambrook and Fritsch, CSH Press, 2001, and Ausubel F. et al., Current Protocols, Wiley Interscience 2005.

In another embodiment, the invention provides compositions, in particular therapeutic and/or pharmaceutical compositions for use as medicaments and/or vaccines in therapeutic methods for the treatment of viral infections or cancer in mammals. The invention teaches the use of TEIPP epitopes, in particular of peptides comprising TEIPP epitopes or nucleic acid vectors encoding the TEIPP epitopes according to the invention, for the manufacture of a medicament capable of eliciting an immune response, preferably a T-cell response, against TAP impaired or deficient cells, which may be TAP impaired or deficient tumour cells or TAP deficient or impaired virally infected and/or transformed cells. The compositions may be used as peptide vaccines or DNA vaccines for eliciting a protective or curative immune response, in particular a T cell response, against virally infected and/or tumor cells.

Formulation of medicaments, ways of administration and the use of pharmaceutically acceptable excipients are known and customary in the art and for instance described in Remington; The Science and Practice of Pharmacy, 21^{nd} Edition 2005, University of Sciences in Philadelphia. Pharmaceutical compositions and medicaments of the invention may thus comprise binders such as lactose, cellulose and derivatives thereof, polyvinylpyrrolidone (PVP), humectants, disintegration promoters, lubricants, disintegrants, starch and derivatives thereof, sugar solubilizers, immuno-stimulatory adjuvants or other excipients. The invention provides methods and means to formulate and manufacture new medicaments and/or pharmaceutical formulations for the treatment of viral infections and tumors or neoplasias comprising TAP impaired or TAP deficient cells, and more in particular for raising immune responses against tumor antigens or viral antigens, such as but not limited to HPV induced malignancies, in particular those induced high risk HPV strains.

Compositions according to the invention for eliciting an immune response in a subject comprise at least one peptide according to the invention, the peptide comprising a TEIPP epitope identified according to the methods disclosed herein. The compositions may optionally comprise one or more peptides comprising a tumor or virus specific epitopes and/or an immune stimulating adjuvant.

In one embodiment the epitope comprising peptides to be admixed in a composition according to this invention are selected from Human Papilloma Virus, in particular from HPV early antigens E2, E6 and E7. Such the peptides may be added to the TEIPP comprising peptide medicament according to the invention. The composition may be used to elicit a T cell response against HPV infected cells, for the treatment of HPV infections and/or HPV induced malignancies.

Advantageously the pharmaceutical composition according to the invention may additionally comprise one or more adjuvants. These adjuvants may be admixed to the pharmaceutical composition according to the invention or may be administered separately to the mammal or human to be treated. Particularly preferred are those adjuvants that are known to act via the Toll like receptors. Immune modifying compounds that are capable of activation of the innate immune system, can be activated particularly well via Toll like receptors (TLR's), including e.g. TLR's 1 to 10. Compounds capable of activating TLR receptors and modifications and derivatives thereof are documented in the art. TLR1 may be activated by bacterial lipoproteins and acetylated forms thereof, TLR2 may in addition be activated by Gram positive bacterial glycolipids, LPS, LPA, LTA, fimbriae, outer membrane proteins, heatshock proteins from bacteria or from the host, and Mycobacterial lipoarabinomannans. TLR3 may be activated by dsRNA, in particular of viral origin, or by the chemical compound poly(I:C). TLR4 may be activated by Gram negative LPS, LTA, Heat shock proteins from the host or from bacterial origin, viral coat or envelope proteins, taxol or derivatives thereof, hyaluronan containing oligosaccharides and fibronectins. TLR5 may be activated with bacterial flagellae or flagellin. TLR6 may be activated by mycobacterial lipoproteins and group B Streptococcus heat labile soluble factor (GBS-F) or Staphylococcus modulins. TLR7 may be activated by imidazoquinolines. TLR9 may be activated by unmethylated CpG DNA or chromatin-IgG complexes. In particular TLR3, TLR7 and TLR9 play an important role in mediating an innate immune response against viral infections, and compounds capable of activating these receptors are particularly preferred for use in the methods of treatment and in the compositions or medicaments according to the invention. Particularly preferred adjuvants comprise, but are not limited to, dsRNA, poly(I:C), unmethylated CpG DNA which trigger TLR3 and TLR9 receptors.

The administration of peptides comprising TEIPP epitopes, or compositions or vaccines comprising these, in order to elicit a T-cell response, in particular a CD4+ T cell response, may be combined with the administration of CD40 receptor and/or 4-1-BB receptor activating compounds or agonists. These may be selected from known compounds, such as various natural or synthetic ligands of these receptors and/or (agonistic) antibodies or fragments and derivates thereof, as described in WO 99/61065 and WO 03/084999, in order to enhance and/or prolong an immune response of peptide vaccination by the activation of dendritic cells, which will aid in the building up of CTL response. The use of CD40 and/or 4-1-BB receptor activating compounds or agonists is therefore particularly preferred. These compounds may be admixed to the pharmaceutical composition according to the invention or may be administered separately.

### DESCRIPTION OF THE FIGURES

FIG. 1. RMA-S.B7-1 Induced T Cells Selectively Recognize and Eradicate TAP-Deficient Cells (a-f)

The response of two independent TEIPP-specific T cell lines against RMA-S and TAP1−/− B cell blasts was measured in cytotoxicity (a-c) and IFNγ release (d-f) assay. RMA-specific control CTL (c and f) killed TAP1−/− blasts only when exogenously loaded with the cognate peptide (16). One out of four comparable experiments is shown. (g) B6 mice were inoculated with TAP-deficient RMA-S tumor cells. Mice were treated with TEIPP-specific T cells (i.v.) together with a single depot of IL-2 (n=12). Control mice were treated with IL-2 alone (n=10). No changes were observed after the last day depicted. The difference between the two groups was statistically significant (Kaplan-Meijer, p=0.0165).

FIG. 2. TEIPP-Specific CTL React Against TAP-Deficient Non-Hematopoietic Cells

(a) TEIPP-specific CTL were incubated with mAb against CD3, CD4 or CD8 before incubation with RMA-S target cells, in order to block TCR signaling. IFNγ production was measured after 6 h. Means of triplicate wells are depicted from one out of three comparable experiments.
(b-c) TEIPP-specific CTL were incubated with fibrosarcoma cells from TAP1−/− or TAP−/−β2m−/−− mice (b), or mouse embryo cells (MEC) from TAP1−/− or wildtype mice (c). All T cell cultures that were raised against RMA-S.B7-1 exhibited comparable specificity. One representative experiment out of four is shown. Means of triplicate wells are depicted.

FIG. 3. Impairment of MHC Class I Antigen Processing at Several Stages Sensitizes Cells for Recognition by TEIPP-Specific CTL

(a) TEIPP-specific CTL were incubated with a panel of TAP-expressing syngeneic tumor cells (see Materials and Methods for descriptions of tumor cell lines). CTL response against TAP-deficient RMA-S in this experiment was 44 ng/ml.
(b) Pre-treatment of TAP-expressing T-cell lymphoma (RMA) and B-cell lymphoma (786) cells with IFNγ reduced the reactivity of TEIPP-specific CTL, as measured after 6 h. MHC class I surface expression on RMA cells was increased three-fold due to IFNγ pre-treatment (not shown). Pre-treatment of TAP-deficient RMA-S did not alter the CTL recognition nor MHC class I expression (not shown).
Very similar results were obtained with other TEIPP CTL clones. Panels depict means of triplicate wells and one of three experiments is shown. (c-e) TAP-expressing RMA tumor cells were treated with the proteasome inhibitor lactacystin and used as targets for cytolysis by two independent TEIPP-specific CTL (c and d) or control RMA-specific CTL (e). The figure shows the percent specific lysis of triplicate wells. One representative experiment of three is shown. Similar results were obtained with the proteasome inhibitors NLVS and LLnL (not shown). (f-g) TAP-expressing colon carcinoma MC38 (f) and melanoma B16 (g) were loaded with oligo DNA coding a partial antisense sequence of TAP1. Recognition by TEIPP-specific CTL was measured by IFNγ release. One out of three comparable experiments is shown. (h) Tapasin−/− LPS B cell blasts are recognized by TEIPP-specific CTL in a CD8-dependent manner. Spleens of TAP1−/−, Tapasin−/− or wild type B6 mice were cultured with LPS and used as targets for TEIPP-specific CTL. Means of triplicate wells from one out of three comparable experiments is shown.

FIG. 4. The Diverse TEIPP-Specific CTL Repertoire Involves Classical and Nonclassical MHC Class I Molecules (a-d)

Four independently derived TEIPP-specific CTL clones are restricted by different MHC class I molecules. EC7.1 cells, which are MHC class I-loss variants of RMA-S 21, were transfected with single classical or nonclassical MHC class I genes (Kb, Db, Qa-1b and M3). C4.4-25-cells are β2m-negative lymphoma cells. Three independent TEIPP CTL clones displayed a reactivity pattern as depicted in panel (d). The response of CTL clones (a) and (b) against RMA-S could be blocked in the presence of anti-Kb and -Db mAb, respectively (not shown). (e-f) Restoration of MHC class I into B78H1 melanoma cells that are TAP2-, Kb- and Db-deficient variants of B16 22 stimulated the Kb- (e) and Db-restricted TEIPP-CTL (f). Restoration of TAP2 by induction with IFNγ (‘+IFNγ’) or stable expression of the gene (‘+TAP2’) 22 resulted in decreased recognition by CTL. Representative results are shown of at least three experiments.

FIG. 5. Identification of a Db-Presented TEIPP Epitope (a-b)

Kb-restricted (a) and Db-restricted TEIPP-CTL (b) respond to human TAP-deficient T2 cells with stable expression of Kb or Db when exogenously loaded with synthetic peptide libraries. Libraries contained peptides with Kb- or Db-binding motifs, Kb lib: xxxxFxxI/L/M (complexity of approximately 108) and Db lib: xxxxNxxxI/L/M (complexity of approximately 109), where x is random for all amino acids. (c-d) Db molecules from RMA-S cells (1011) were immunoaffinity purified and acid eluted peptides were separated by reversed phase HPLC, applying an increasing acetonitril gradient (OD214 left axis, % acetonitril right axis) (c). HPLC fractions were loaded unto T2.Db cells and TEIPP-specific, Db-restricted CTL were applied to detect the eluted peptide-epitope (d). Comparable results were obtained for four similar peptide elutions. Of note, CTL recognition of HPLC fractions depended on the presence of the relevant class I molecule on target cells (not shown). (e) MCLRMTAVM peptide is processed and presented from the Trh4 gene. The Trh4 gene was cloned from RMA-S cells using gene-specific primers. Trh4 gene in the reversed orientation (Trh4r) served as control plasmid. Hela cells expressing the viral TAP inhibitor ICP47 were transiently transfected with Trh4 or Trh4r in combination with H-2 Db or -Kb encoding plasmids. Db-restricted TEIPP CTL were applied to detect proper processing and MHC class I-restricted presentation of the MCLRMTAVM peptide. Similar IFNγ release by CTL was observed in an additional experiment. (f-g) Synthetic peptides SLSRLSGTV (circles), retrieved from the peptide library, and MCLRMTAVM (squares) derived from the Trh4 gene were tested in titrating amounts for CTL recognition by the Db-restricted TEIPP CTL (f). HPLC profile of the synthetic MCLRMTAVM peptide (g) using the same acetonitril gradient as (c). The early peptide peak in the profile contains the MCLRMTAVM sequence of which the C-terminal methionine is oxidized.

FIG. 6. The MCLRMTAVM Peptide-Epitope is Present in HPLC Fractions Containing Db-Binding Peptides from RMA-S

Db molecules from 1011 RMA-S cells were purified and binding peptides were separated by HPLC. The peptide fraction that sensitized Db-restricted TEIPP CTL was subjected to tandem MS selecting the ion MH+ 1055.49, corresponding to the mass of MCLRMTAVM. Fragmentation spectrum of this ion comprised in Db-eluted peptide fraction from RMA-S is depicted in panel (a) and that of the synthetic MCLRMTAVM peptide in panel (b). Identifiable b and y″ ions are underlined in the peptide sequence in the inlet in (a). Ions in (a) that belong to the MCLRMTAVM peptide are marked with asterisks (*). Diamonds indicate the selection window for MS/MS (♦).

FIG. 7. Organization and Expression of the Trh4 Gene (a-b)

The Trh4 gene gives rise to two mRNA splice variants discernible on the inclusion or exclusion of exon 9a (a). Inclusion of exon 9a results in the generation of long transcripts and in a shift in the reading frame of the succeeding exon 10. The TEIPP CTL epitope MCLRMTAVM is therefore exclusively encoded by long transcripts of Trh4. Of note, the peptide is comprised by the very COOH-terminus of the Trh4 protein due to the presence of an early stop codon in the sequence of the long transcript (b). (c) Short and long transcripts of Trh4 are detected in all cell lines tested indifferent of their TAP-status. Products of RT-PCR using primers as indicated in panel (a) were separated on agarose gels. See materials and methods for description of the used cell lines.

FIG. 8. Immunization with Trh4 Derived TEIPP Peptide Induces Protection Against RMA-S

Groups of B6 mice (n=10) were immunized with synthetic peptides comprising TEIPP Db peptide MCLRMTAVM or control Db peptide SSPVNSLRNVV51 combined with T helper peptide EPLTSLTPRCNTAWNRLKL29 and CpG oligonucleotides 28 and challenged with TAP-deficient RMA-S lymphoma cells. Mice were challenged at day 0 and immunized at days −14, 7 and 14. Immunization with irradiated RMA-S.B7 cells served as positive control. The experiment was repeated once with comparable results.

### EXAMPLES

### Example 1

### Isolation of Clonal T Cell Cultures Capable of Eliminating TAP-Deficient Target Cells In Vitro and In Vivo

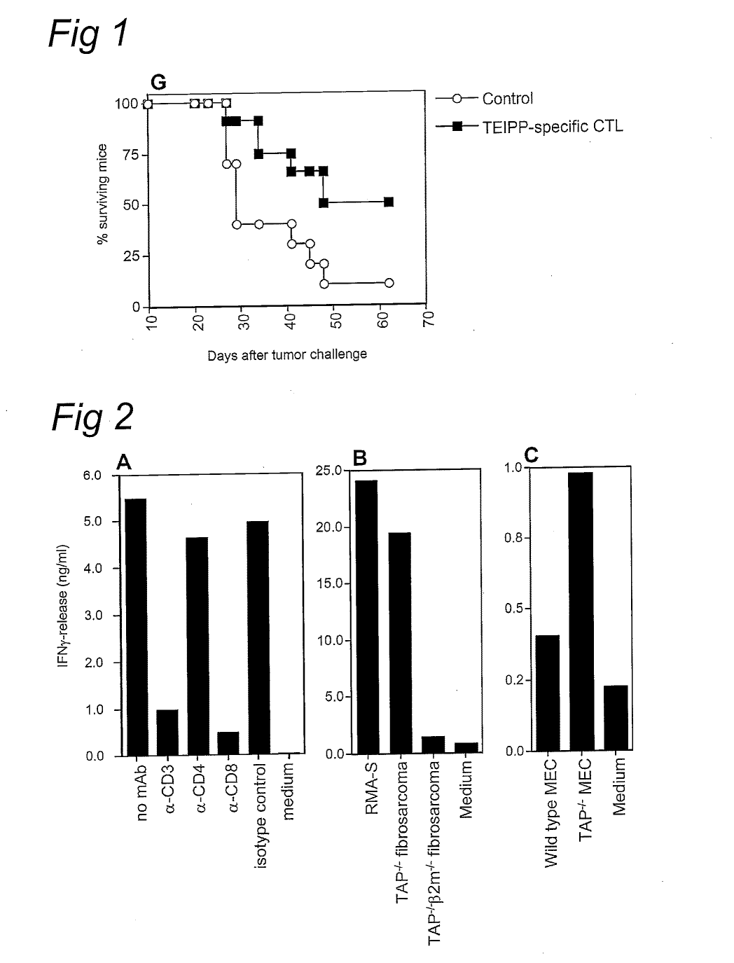

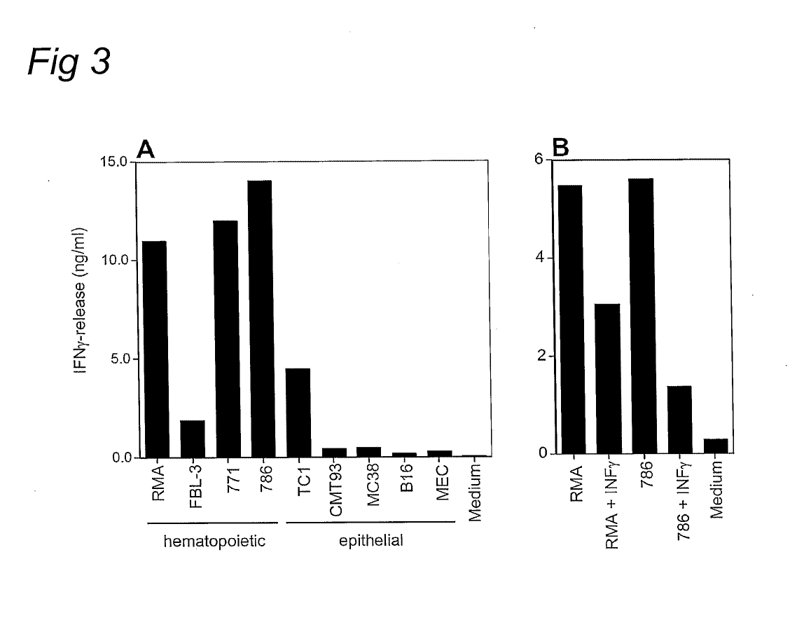

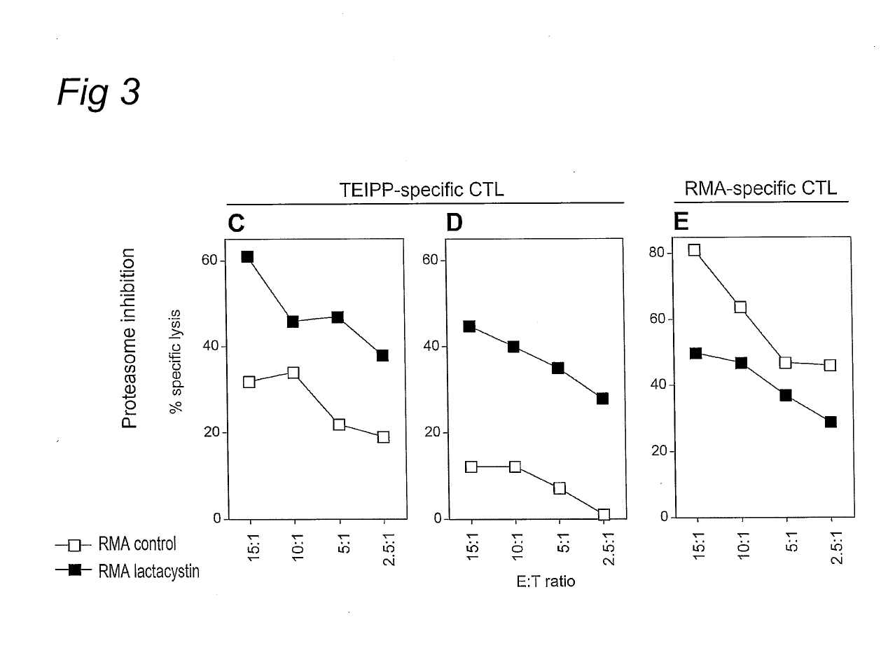

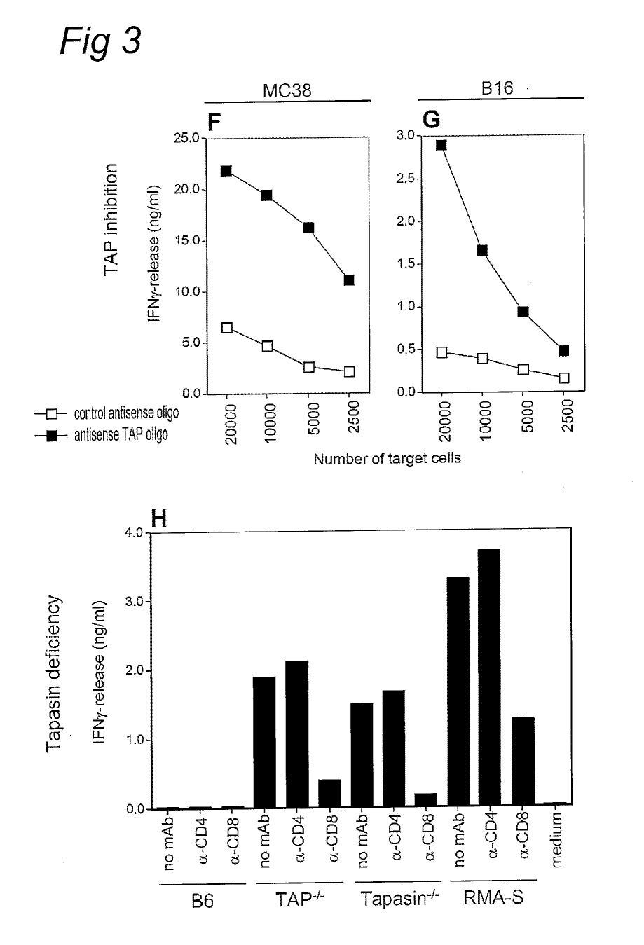

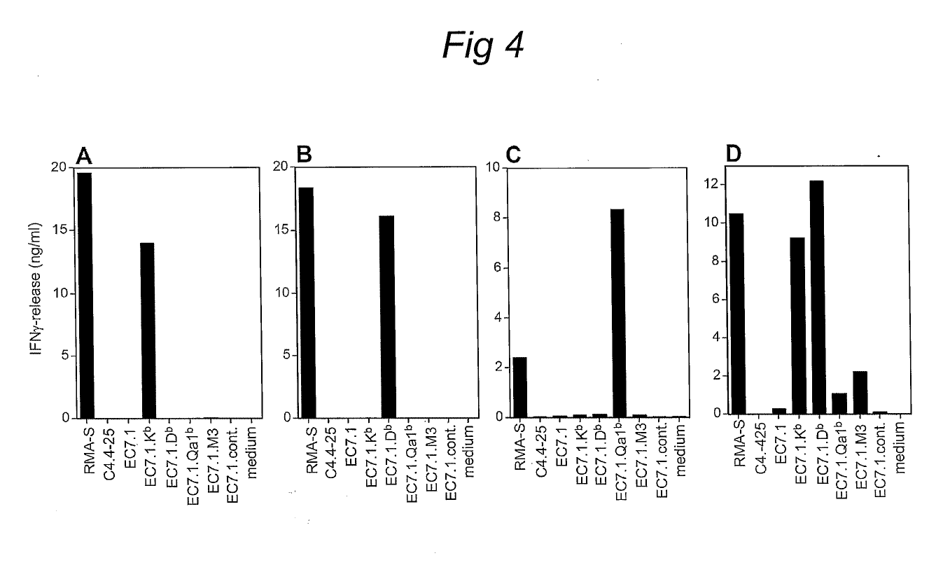

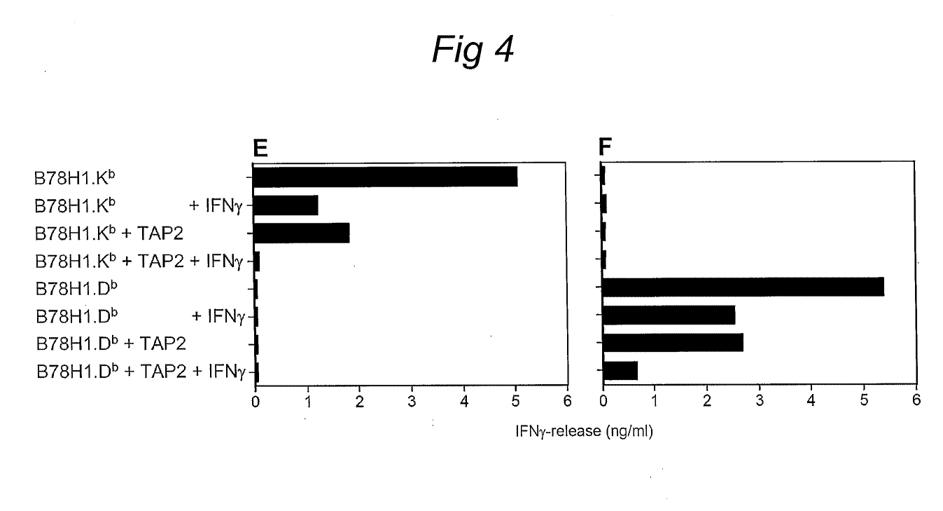

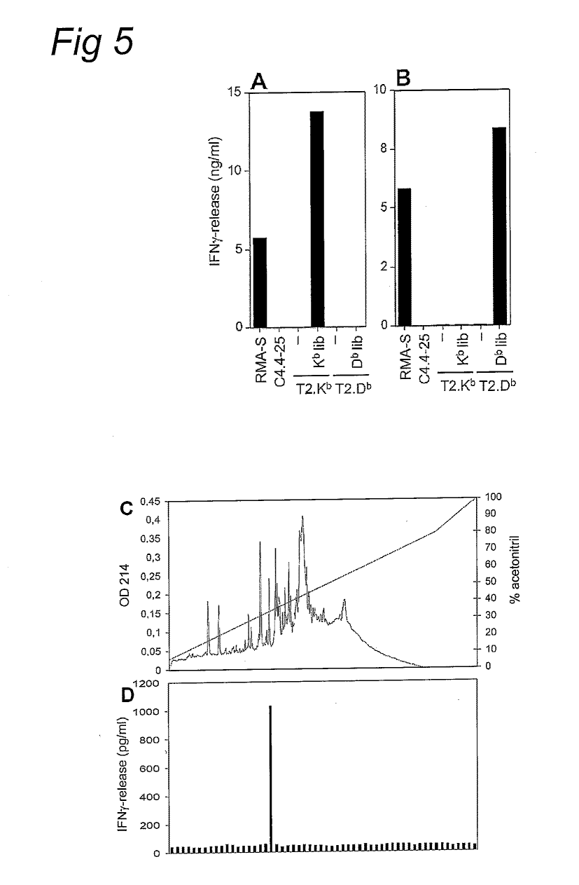

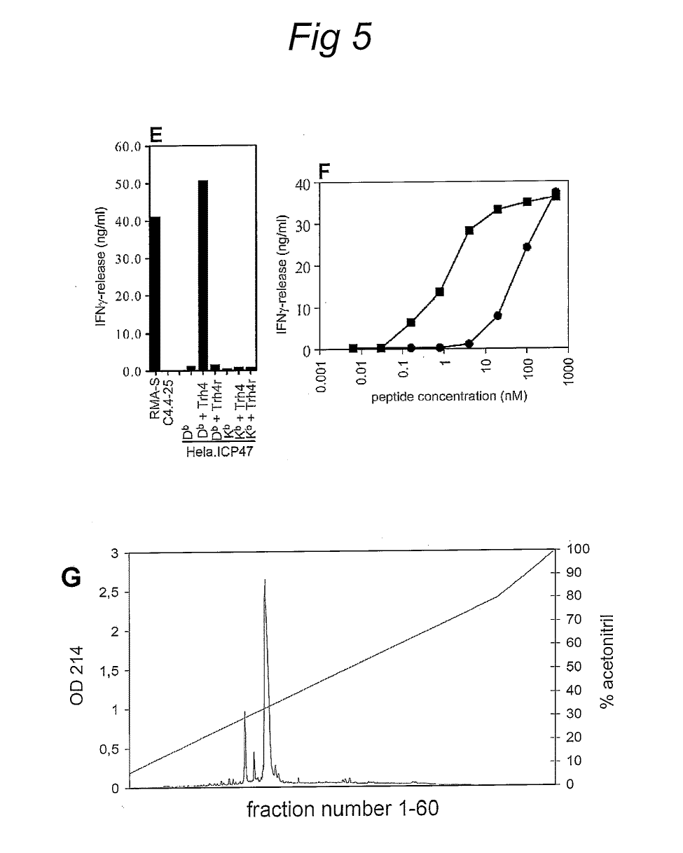

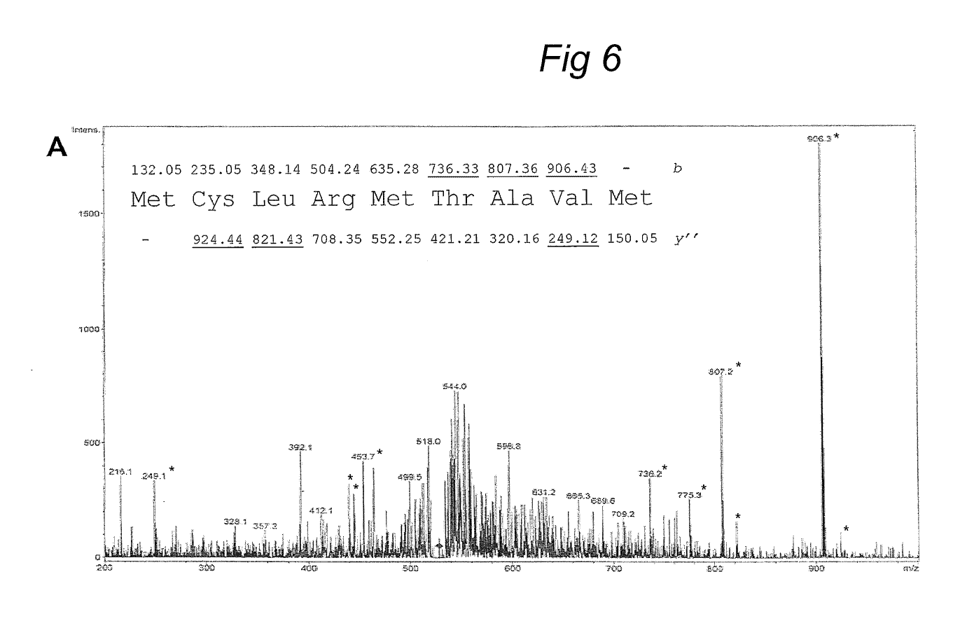

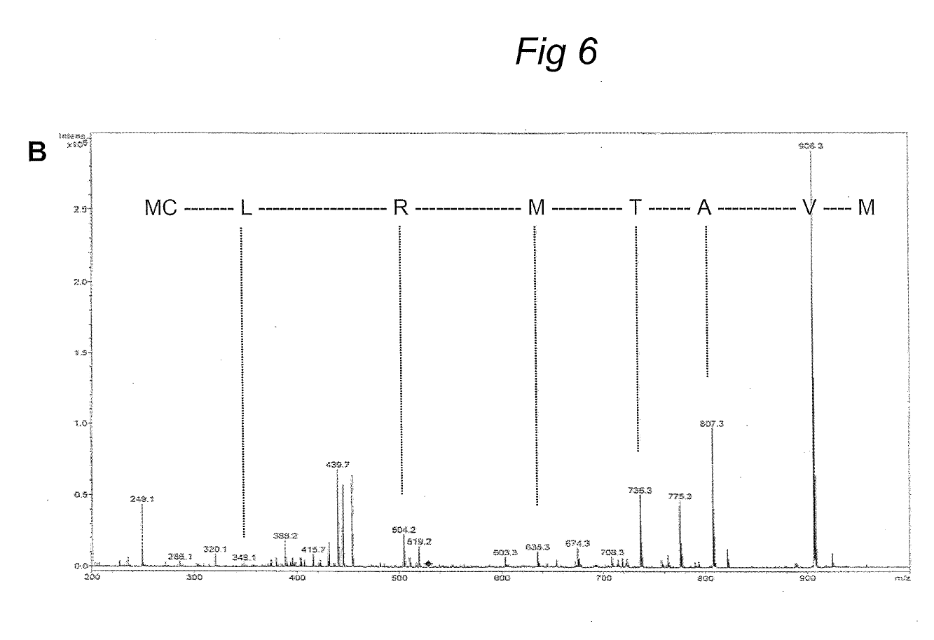

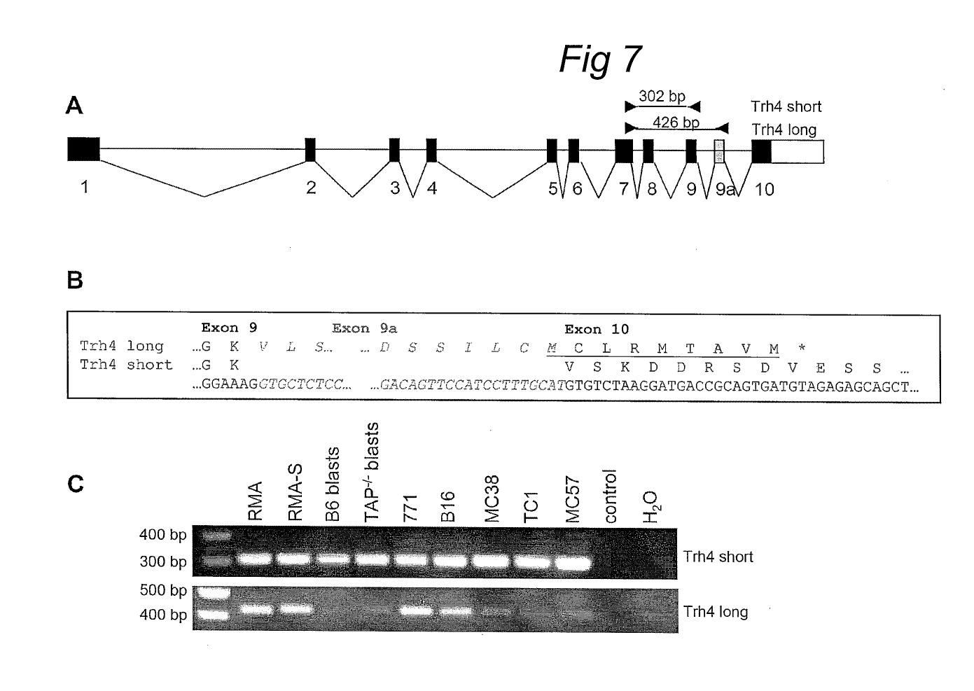

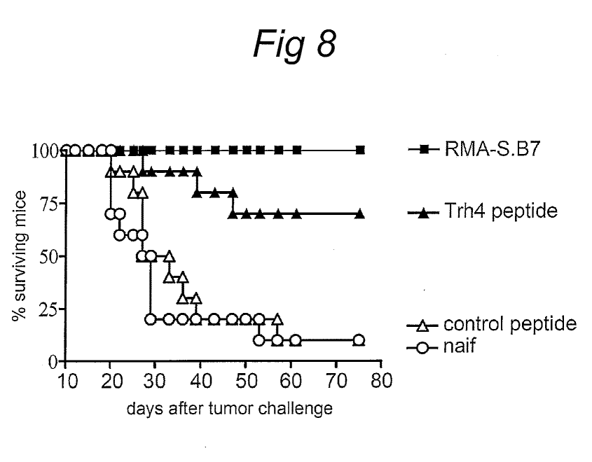

Our previous work pointed at the existence of CD8+ T cells that are capable of reacting against TAP-deficient tumor cells (13). For an in depth analysis of these T cells and their target structure, which we refer to as TEIPP (T cell Epitopes associated with Impaired Peptide Processing), we established long-term T cell cultures from C57BL/6 (B6) mice by in vivo immunization with TAP-deficient RMA-S.B7-1 tumor cells followed by repeated in vitro restimulation. Polyclonal T cell cultures and clonal T cell lines derived thereof displayed strong cytolytic activity and IFN gamma-release against RMA-S cells and B cell blasts derived from TAP1−/− mice, whereas B cell blasts of wild type mice or β2-microglobulin (β2m)-deficient mice were not recognized (FIG. 1a-f). The requirement of β2m expression suggests that the target structure of TEIPP-specific T-cells comprises MHC class I molecules. Furthermore, this target is present on both transformed and non-transformed cells, provided that these cells are TAP-deficient.

Adoptive transfer of TEIPP-specific T-cells into B6 mice that were challenged with a tumorigenic dose of TAP-deficient RMA-S cells revealed that these T-cells can exert a marked anti-tumor effect in vivo (FIG. 1g). The majority of B6 mice that received a challenge with the TAP-deficient RMA-S tumor cells died within 7 weeks due to progressive tumor development. In contrast, a single administration of TEIPP-specific T-cells resulted in rejection of this highly aggressive tumor in half of the mice and delayed tumor development in the other mice. Importantly, histological screening of a range of tissues isolated from successfully treated mice did not reveal any signs of autoimmune damage (data not shown). This indicates that normal somatic tissues, which are TAP-proficient, do not express TEIPP, allowing selective in vivo targeting of the TAP-deficient tumor cells by TEIPP-specific T-cells.

### Example 2

### TEIPP-Specific T Cells Display a Conventional CD8+ CTL Phenotype and Function

In view of the paradoxical finding that TEIPP-specific T cells, similar to NK cells, selectively recognize targets that express very low surface levels of MHC class I, we analyzed the expression of several CTL and NK cell markers at the surface of five independently derived T cell clones. All clones displayed a CD3+, CD4−, CD8+, TCRα/β+ phenotype, while generally lacking expression of common NK cell markers such as NK1.1, CD16 and DX-5. (Table I). TEIPP-specific T cells did express CD94 as well as NKG2A, -C and -D, as determined at the mRNA level, while one of the clones was also positive for transcripts of the Ly49-family (Table I). However, CTL clones directed against ‘conventional’ MHC class I-bound peptides similarly expressed these NK-associated markers (Table I), in accordance with data published by others 14. The phenotype of TEIPP-specific T cells was therefore indistinguishable from that of regular CD8+ CTL. This also applied to T cell function, in that reactivity of TEIPP CTL was strongly inhibited in the presence of anti-CD3 Ab or anti-CD8 Ab (FIG. 2a). Our combined data indicate that, despite their unconventional reactivity pattern, TEIPP-specific T cells are not different from regular CTL and, therefore, may be exploited in immunotherapeutic strategies in a similar manner.

TABLE I
Phenotypic Characterisation of TEIPP-speciffc CTL

| | TEIPP-specific | Conventional | Detection |
|---|---|---|---|
| | CTL clones | CTL clones | method |
| CD3 | 5/5^{a} | 8/8 | flow cytometry^{b} |
| CD4 | 0/5 | 0/8 | flow cytometry |
| CD8 | 5/5 | 8/8 | flow cytometry |
| TCRαβ | 5/5 | 8/8 | flow cytometry |
| TCR Vα3 | 4/5 | N.D.^{c} | flow cytometry |
| TCR Vβ8 | 4/5 | N.D. | flow cytometry |
| TCR Vβ11 | 1/5 | N.D. | flow cytometry |
| NK1.1; | 0/5 | 0/8 | flow cytometry |
| DX-5 | 1/5 | 2/8 | flow cytometry |
| CD16 | 0/5 | 0/8 | flow cytometry |
| Ly49 | 1/3 | 1/7 | RT-PCR^{d} |
| CD94 | 3/3 | 7/7 | RT-PCR |
| NKG2A | 3/3 | 5/7 | RT-PCR |
| NKG2C/E | 3/3 | 6/7 | RT-PCR |
| NKG2D | 3/3 | 7/7 | RT-PCR |

^{a}number of positive CTL clones/total number tested
^{b}see material and methods for applied mAb and primer sequences
^{c}N.D.—not determined
^{d}using generic Ly49 PCR primers, no surface expression detected with available mAbs

### Example 3

### TEIPP-Specific CTL Detect Deficiencies at Multiple Levels of the MHC Class I Antigen-Processing Pathway in Cells of Diverse Histological Origin

Thus far, only cells of hematopoietic origin had been tested against TEIPP-specific CTL. However, TEIPP-expression is not restricted to cells of hematopoietic origin, in that a fibrosarcoma tumor cell line of TAP1−/− origin was efficiently recognized, whereas a fibrosarcoma from a TAP1−/−β2m−/− background did not trigger these CTL (FIG. 2b). Likewise, TEIPP-specific CTL recognized immortalized mouse embryo fibroblasts from TAP1−/− origin (FIG. 2c). These data indicated that the TEIPP target structure is expressed by cells of hematopoietic and non-hematopoietic origin, provided that these cells lack TAP-function but do have expression of β2m. Rather unexpectedly, we found that expression of TEIPP also extends to a selection of TAP-expressing cells. For instance, RMA, the TAP-expressing counterpart of RMA-S against which these T cells were raised, was also recognized by TEIPP-directed CTL, albeit to lower extent (FIG. 3a). Furthermore, the TEIPP-specific CTL recognized two independent TAP-expressing murine leukemia virus-induced B cell lymphomas (FIG. 3a, lines 771 and 786) and, of the tumor targets of non-hematopoietic origin, HPV type 16-transformed fibroblasts (FIG. 3a, line TC1). All these tumor cells expressed TAP, as illustrated by the fact that they serve as proper targets for control CTL that react against defined, TAP-dependent epitopes 15, 16. We therefore considered the possibility that partial deficiencies in MHC class I antigen processing may account for the recognition of several TAP-expressing murine tumor cells by TEIPP-specific CTL. Such partial defects are commonly found in human cancers 5. We examined this idea by treatment of RMA cells with IFNγ, which is known to enhance MHC class I antigen processing at multiple levels, including the transcription of TAP 17. Indeed, we found that pretreatment of RMA with IFNγ reduced recognition of these cells by TEIPP-specific CTL (FIG. 3b). In contrast, recognition by RMA-specific control CTL was increased (data not shown). A similar effect of IFNγ was found with respect to the recognition of lymphoma B-cells (FIG. 3b, line 786). Treatment of RMA-S, which harbors a genetic defect in TAP2 18, with IFNγ did not result in an increased recognition (data not shown). Taken together, our data indicate that even partial deficiencies in antigen processing can sensitize tumor cells for recognition by TEIPP-specific CTL.

In order to make an inventory of the deficiencies that can result in TEIPP expression, we assessed the effect of several targeted interventions in the MHC class I processing pathway. The first step of the class I antigen processing pathway involves proteasome-mediated degradation of proteins into peptides, which are subsequently transported into the ER by TAP. Inhibition of proteasome activity limits the availability of peptides for TAP transport 19, and thus creates a situation comparable to impaired TAP-function. We therefore investigated the effect of specific proteasome inhibitors on recognition of TAP-positive target cells by TEIPP-specific CTL. Indeed, treatment of RMA cells with lactacystin increased their sensitivity to lysis by these CTL (FIGS. 3c and d). As expected, RMA recognition by control CTL was decreased (FIG. 3e). Secondly, a partial deficiency in TAP-function was induced by treating MC38 colon carcinoma cells and B16 melanoma cells, which were otherwise not recognized by TEIPP-specific CTL (FIG. 3a), with TAP1-specific anti-sense oligonucleotides. This treatment resulted in increased recognition by TEIPP-specific CTL (FIGS. 3f and g). The outcome of this experiment supports the hypothesis that a partial deficiency in TAP-function can result in recognition of tumor cells by TEIPP-specific CTL. Furthermore, it confirms that also tumors of non-hematopoietic origin can be targeted by these CTL, provided that they harbor a deficiency in TAP-dependent antigen-processing (compare FIG. 3a with 3f and g). Finally, a deficiency in tapasin, a chaperone protein involved in TAP-mediated peptide loading of MHC class I molecules 20, also resulted in recognition of target cells by TEIPP-specific CTL (FIG. 3h). This recognition could be inhibited by Ab specific for CD8, supporting the involvement of the TCR in target recognition (FIG. 3h).

In conclusion, deficiencies at various levels of the MHC class I antigen-processing pathway result in the sensitisation of cells of diverse histological origin for recognition by TEIPP-specific CTL.

### Example 4

### Recognition by TEIPP-Specific CTL is Restricted by Classical and Non-Classical PNC Class I Molecules

Even though class I molecules are expressed at very low levels on antigen processing-deficient cells, the requirement for β2m expression by target cells (FIG. 1a-f and 2b) suggested that TEIPP-specific CTL were MHC class I restricted. This issue was investigated with the use as target cells of a class I negative variant of the TAP-deficient RMA-S cells, designated EC7.1 21. Testing of six independently derived TEIPP CTL clones against a panel of EC7.1 transfectants expressing defined classical and nonclassical MHC class I genes revealed that these CTL, which exhibit otherwise indistinguishable specificities and phenotypes (FIG. 1-3 and Table I), displayed distinct MHC restriction patterns (FIG. 4a-d). Three of the TEIPP-specific CTL clones exhibited reactivity against target cells positive for Kb, Db or Qa-1b, respectively (FIG. 4a-c), whereas the other three CTL clones recognized multiple EC7.1 lines expressing either the classical MHC class I molecules Kb or Db, or the non-classical MHC class I molecules Qa-1b or M3 (FIG. 4d). In all cases, recognition could be inhibited by anti-CD3 or -CD8 mAb, but not by control anti-CD4 in Ab, indicating that the TCR was critically involved in the interaction with the various class I molecules, even in the case of the TEIPP CTL that exhibited broadly restricted reactivity (online supplementary data Fig. S1). For the Kb- and Db-restricted TEIPP CTL, comparable results were obtained using transfectants of a TAP- and class I-negative variant of the B16 melanoma, B78H1 22 (FIG. 4e-f). Notably, restoration of TAP function in these cells by gene transfer or by IFNγ treatment led to a decreased TEIPP CTL recognition. The combination of TAP2 gene transfer and IFNγ treatment abolished recognition (FIG. 4e-f). This confirms that presentation of TEIPP requires an at least partially impaired antigen processing (see also FIG. 3). In conclusion, the TEIPP CTL repertoire appears to comprise a wide variety of specificities that is restricted by different classical and nonclassical MHC class I molecules.

### Example 5

### The TEIPP Target Structure Comprises MHC Class I-Bound Peptides

The Kb- and Db-restricted TEIPP CTL, which reacted against murine EC7.1 and B78H1 cells expressing the correct MHC class I molecule (FIG. 4a-b and e-f), failed to recognize human TAP-deficient T2 cells expressing Kb or Db (FIG. 5a-b). Because HLA-A*0201 molecules at the surface of T2 cells were found to be loaded with peptides derived from human proteins 23, 24, the Kb and Db molecules expressed on T2 cells are expected to be similarly loaded with such peptides. This suggests that recognition by TEIPP-specific CTL depends on peptides derived from murine proteins that are not sufficiently conserved between mouse and human and, furthermore, exclude that such peptides could be of artificial origin, such as the tissue culture medium. Direct proof for the recognition of peptides by the Kb- and Db-restricted CTL clones was provided by the finding that synthetic peptide libraries based on the Kb- or Db-binding-motifs selectively sensitized T2 cells for recognition by the respective TEIPP-CTL (FIG. 5a-b).

These results imply that the processing-deficient cells recognized by these CTL present a unique, so far unexplored repertoire of peptide antigens. Since unraveling the molecular nature of the TEIPP peptide repertoire would provide fundamental insight to the basis of their immunogenicity and, moreover, would permit the development of peptide-based vaccine modalities for the treatment of ‘escaped’ tumors, we embarked on the elution of naturally processed peptides from affinity-purified Kb and Db molecules from RMA-S cells. We observed reactivity of TEIPP-directed CTL against distinct fractions of HPLC separated peptides, as shown for the Db-restricted TEIPP CTL in FIGS. 5c and d. Similar results were obtained for the Kb-restricted CTL clone. Kb eluted peptides failed to sensitize Db-expressing target cells and vise versa, indicating that these class I molecules harbor distinct peptide repertoires. Mass spectrometry analysis of HPLC peptide fractions demonstrated at least 40 different peptide-masses that were selectively present in the CTL recognized fractions. Although the yield of peptides from large quantities of RMA-S cells (1011) was very low (on average 2 fmol per component), the sequences of the 3 most abundant peptides from Kb were identified by state of the art tandem mass spectrometry. However, corresponding synthetic peptides were not recognized by the Kb-restricted TEIPP CTL (data not shown). Recovery of Db-eluted peptides was too low, and the diversity of peptide masses to large, to permit systematic identification of peptide sequences.

### Example 6

### Identification of a Db-Presented TEIPP Epitope

In view of the technical difficulties described above, we pursued the identification of TEIPP with a novel approach that combined screening of synthetic peptide libraries with information from eluted natural peptides and with database search. Db-restricted TEIPP CTL were used to screen a synthetic bead-assisted peptide library with a complexity of approximately 6.5×10⁵ different 9-mer peptides. This resulted in the identification of peptide mimotope SLSRLSGTV (FIG. 5f). A collection of 171 single peptides was then generated in which each amino acid of this mimotope sequence was exchanged for all other amino acids. Measurement of CTL reactivity against this peptide collection revealed that some exchanges completely destroyed recognition, whereas others strongly improved the recognition. All amino acid substitutions that led to equal or improved CTL recognition were combined to formulate a ‘TCR motif pattern’ (Table II). Pattern search in the EMBL mouse protein index database (http://www.ebi.ac.uk/IPI/IPIhelp.html) yielded 77 naturally occurring 9-mer peptides matching this motif. To determine which of these 77 candidates constitutes the naturally processed epitope recognized by our Db-restricted CTL, we applied three selection criteria. First, the peptides should trigger the CTL at low concentrations. Of the 77 tested peptides, 13 were found to elicit CTL activity at concentrations of 250 pM or lower. Secondly, the retention time on HPLC of the candidate peptides should match that of the naturally eluted peptide. This criterion was fulfilled by 6 of the 13 remaining candidates. Finally, the cDNAs encoding these 6 peptides were cloned and transfected into TAP-inhibited recipient cells to examine proper processing and presentation to the CTL. Only one cDNA conferred CTL recognition: the Trh4 gene encoding an ER-membrane spanning protein that is a member of the TLC family of fatty acid regulators (TLC-TRAM, LAG1 and CLN8; acc. nr. BC043059) 25, 26.

TABLE II
TCR motif pattern^{a}
Amino acid position

| 1 | 2 | 3 | 4 | 5 | 6 | 7 | 8 | 9 |
|---|---|---|---|---|---|---|---|---|
| X^{b} | A | I | R | L | S | A | T | A |
| | C | L | K | M | T | G | V | I |
| | G | M | | N | | I | | L |
| | I | N | | | | S | | M |
| | L | P | | | | V | | V |
| | M | Q | | | | | | |
| | Q | R | | | | | | |
| | R | S | | | | | | |
| | S | V | | | | | | |
| | T | W | | | | | | |
| | V | | | | | | | |
| | Y | | | | | | | |
| S^{c} | L | S | R | L | S | G | T | V |
| M | C | L | R | M | T | A | V | M |

^{a}Amino acid substitutions that resulted in equal or increased CTL recognition from supplementary Table II are here summarized.
^{b}represents all amino acids.
^{c}SLSRLSGTV, D^{b} peptide mimotope MCLRMTAVM, revealed D^{b} epitope

The conclusion that the Trh4 gene encodes the natural epitope of the Db-restricted TEIPP-specific CTL clone is firmly based on the following five experimental results. Firstly, as mentioned above, transfection of the Trh4 cDNA selectively sensitized Db-expressing targets for CTL recognition (FIG. 5e). Secondly, the 9-mer peptide MCLRMTAVM comprised by the Trh4 protein is efficiently recognized by the Db-restricted TEIPP CTL, at much lower concentration than the mimotope peptide that was selected from the synthetic peptide library (FIG. 5f). Thirdly, the HPLC retention time of the MCLRMTAVM peptide matched that of the natural Db-restricted epitope as eluted from RMA-S (FIG. 5g, compare with FIGS. 5c and d). Fourthly, the acquired knowledge of the peptide sequence and its corresponding mass allowed us to trace this mass in the HPLC fraction that was recognized by the Db-restricted CTL. The fragmentation spectrum of this mass (MH+ 1055.49), as obtained by tandem mass spectrometry, matched exactly with that of the synthetic peptide with the sequence MCLRMTAVM (FIG. 6). The fifth critical piece of data was the position of the MCLRMTAVM peptide in the Trh4 protein. The GenBank lists two alternatively spliced Trh4 gene transcripts (FIG. 7a). Inclusion of exon 9a into the longer Trh4 transcript leads to a frame shift and early termination of translation in exon 10. The key consequence of this frame shift is that the resulting protein comprises the peptide MCLRMTAVM as its C-terminus (FIG. 7b). Because the C-terminal domain of the Trh4 protein protrudes into the ER lumen (25), this TEIPP peptide is expected to be processed into MHC class I in a proteasome and TAP-independent manner, as described previously by others (27).

Detection of Trh4 mRNA expression by RT-PCR revealed that both splice variants are widely expressed in transformed and non-transformed cells, in line with the general metabolic function of the Trh4 gene products (FIG. 7c). Notably, the TEIPP-encoding long Trh4 transcript is expressed by both TAP-proficient and TAP-deficient cells. It is therefore conceivable that the epitope MCLRMTAVM is available in the ER of all cells, but that only in processing deficient cells it gains access to MHC class I molecules.

### Example 7

### Peptide Vaccination Induces Protective Anti-Tumor CTL

Vaccination with synthetic peptides comprising tumor-specific T-cell epitopes have been shown to confer protective anti-tumor immunity in various murine tumor models (2, 28). We therefore immunized groups of mice with the newly identified Db-restricted TEIPP CTL epitope, or a control CTL epitope, in combination with a previously identified tumor-specific CD4+ T-helper epitope (29), and challenged the mice with a tumorigenic dose of RMA-S cells. Naïve mice and mice that were immunized with control peptide developed progressively growing tumors, whereas immunization with the Trh4 derived peptide led to tumor protection in the majority of mice (FIG. 8). These data demonstrate that TEIPP can be applied effectively in peptide-based vaccines for CTL-mediated targeting of processing-deficient tumors.

### Example 8

### Identification of Human Epitopes Differentially Expressed on TAP Deficient and TAP Proficient Cells

In order to identify TAP-independently presented peptides and to study their relative abundance on TAP^{-/-} cells in comparison to TAP^{+/+} cells we used the human B-cell lines LCL721.174 (TAP^{-/-}) and LCL721.45 (TAP^{+/+}). Here we sequenced more than 40 peptides from LCL721.174 cells presented by HLA-A*02 and -B*51. The MHC presentation level of these peptides was quantitatively compared between LCL721.174 cell line and its TAP-expressing progenitor cell line LCL721.45 using mass spectrometry after differential isotope labelling. Additionally peptides specifically presented on TAP^{-/-} cells (first 33 sequences with a 721.174/721 ratio>1) were positively tested for their HLA binding via MHC refolding. Many signal sequence derived peptides (SSP) have been identified as TAP-independently presented on HLA-A*02 of LCL721.174 cells, table III below. But interestingly we identified apart from SSP also several non signal sequence derived, TAP-independently presented peptides. The peptides identified in the table below will be used to assay TEIPP specific T-cells to identify TEIPP specific T cell lines. The sequences identified will also allow raising of CTL immune responses, optionally by modifying the peptides. The identified HLA ligands are suitable to be used for immunotherapeutic treatment of various human TAP-deficient tumors.

TABLE III
SEQ ID No's 3 to 35 in the sequence listing correspond to the 33 TEIPP
sequences of table III with a 721.174/721 ratio > 1.

| | | | | | | |
|---|---|---|---|---|---|---|
| | | | AA | signal | HLA | 721.174/ |
| Sequence | Gene Symbol | Gene | position | seq | type | 721 ratio |

| | | | | | | |
|---|---|---|---|---|---|---|
| LLSAEPVPA | CD79B | CD79B antigen | 20-28 | 28\|29 | A02 | 556.8 |
| LLGPRLVLA | TMP21 | transmembrane trafficking protein | 22-31 | 31\|32 | A02 | 253.2119309 |
| SLWGQPAEA | COL4A5 | collagen, type IV, alpha 5 | 18-26 | 26\|27 | A02 | 124.4382022 |
| VLAPRVLRA | RCN1 | reticulocalbin 1, EF-hand calcium | 21-29 | 29\|30 | A02 | 120.2890355 |
| | | binding domain | | | | |
| ALVVQVAEA | HEXB | hexosaminidase B | 34-42 | 42\|43 | A02 | 116.125 |
| HGVFLPLV | KIAA0247 | KIAA0247 | 21-28 | 39\|40 | B51 | 92.33678756 |
| LLAAWTARA | APP | amyloid beta A4 precursor protein | 9-17 | 17\|18 | A02 | 91.92473118 |
| VLLKARLVPA | KIAA1946 | KIAA1946 | 19-28 | 28\|29 | A02 | 47.71328671 |
| KMDASLGNLFA | FAM3C | family with sequence similarity 3, | 30-40 | 24\|25 | A02 | 36.39147287 |
| | | member C | | | | |
| LLFSHVDHVIA | SLC8A1 | solute carrier family 8, member 1 | 25-35 | 35\|36 | A02 | 28.09705882 |
| | | LDL receptor-related protein | | | | |
| FLGPWPAAS | LRPAP1 | associated protein 1 | 22-30 | 32\|33 | A02 | 23.6935867 |
| | | vitamin K epoxide reductase | | | | |
| SLYALHVKA | VKORC1 | complex, subunit 1 | 23-31 | 31\|32 | A02 | 23.10884354 |
| LLLSAEPVPA | CD79B | CD79B antigen | 19-28 | 28\|29 | A02 | 22.24096386 |
| | | proline-, glutamic acid-, leucine-rich | | | | |
| AMAPPSHLLL | PELP1 | protein 1 | 473-482 | 21\|22 | A02 | 18.29381443 |
| MAPLALHLL | IL4I1 | interleukin 4 induced 1 | 1-9 | 21\|22 | B51 | 17.95561798 |
| FLLGPRLVLA | TMP21 | transmembrane trafficking protein | 22-31 | 31\|32 | A02 | 17.85238095 |
| LLLDVPTAAV | IFI30 | interferon, gamma-inducible protein | 15-24 | 26\|27 | A02 | 17.80829016 |
| | | 30 | | | | |
| LLLDVPTAAVQA | IFI30 | interferon, gamma-inducible protein | 15-26 | 26\|27 | A02 | 14.7751073 |
| | | 30 | | | | |
| LLLDVPTAA | IFI30 | interferon, gamma-inducible protein | 15-23 | 26\|27 | A02 | 14.7 |
| | | 30 | | | | |
| LLIJVPTAAV | IFI30 | interferon, gamma-inducible protein | 16-24 | 26\|27 | A02 | 14.38864307 |
| | | 30 | | | | |
| VLFRGGPRGLLAVA | SSR1 | signal sequence receptor, alpha | 19-32 | 20\|21 | A02 | 12.84439834 |
| ALLSSLNDF | NIF3L1 | NIF3 NGG1 interacting factor 3-like | 5-13 | no | A02 | 12.84277344 |
| | | 1 | | | | |
| IITKEVLAP | EST Sequence | QV3-HT1016-221100-480-e10 | frame 3 | n.d. | A02 | 12.77545692 |
| | | HT1016 | | | | |
| QLQEGKNVIGL | TAGLN2 | transgelin 2 | 166-176 | no | A02 | 7.893867925 |
| | | transitional epithelia response | | | | |
| ILAPAGSLPKI | TEREl | protein | 328-336 | no | B51 | 6.21086262 |
| | | calcium binding protein Cab45 | | | | |
| MASRWGPLIG | Cab45 | precursor | 8-17 | 36\|37 | B51 | 4.549261084 |
| AVLALVLAPAGA | NRP1 | neuropilin 1 | 10-21 | 21\|22 | A02 | 4.314705862 |
| LAPRVLRA | RCN1 | reticulocalbin 1, EF-hand calcium | 22-29 | 29\|30 | A02 | 4.213924051 |
| | | binding domain | | | | |
| AALLDVRSVP | GDF5 | growth differentiation factor 5 | 275-284 | 27\|28 | A02 | 3.82848392 |
| SLPKKLALL | HSPC023 | HSPC023 protein | 72-80 | no | A02 | 3.242718447 |
| KAPVTKVAA | PDLIM1 | PDZ and LIM domain 1 | 241-249 | no | B51 | 1.748652291 |
| AMAQLLLVL | EST Sequence | 602495863F1 NIH_MGC_75 | Frame 6 | n.d. | A02 | 1.676804813 |
| NPLPSKETI | TMSB4X | thymosin, beta 4, X-linked | 27-35 | no | B51 | 1.224215825 |
| MFPLVKSAL | COX7B | cytochrome c oxidase subunit VIIb | 1-9 | no | B51 | 0.903678422 |
| | | major histocompatibility complex, | | | | |
| MAPRTLVL | HLA-A | class I, A | 4-11 | 24\|25 | B51 | 0.523246841 |
| TLLGHEFVL | CDC27 | Cell division cycle 27 | 605-613 | no | A02 | 0.410165485 |
| LPHVPLGVI | AUP1 | ancient ubiquitous protein 1 | 374-382 | 37\|38 | B51 | 0.300135808 |
| YLTAEILEL | HIST1H2AJ | histone 1, H2aj | 58-66 | no | A02 | 0.274565115 |
| LPREILNLI | LOC116064 | hypothetical protein LOC116064 | 259-267 | no | B51 | 0.274481428 |
| LLDRFLATV | CCNI | cyclin I | 72-80 | no | A02 | 0.210191083 |
| GSHSMRYF | HLA-A, -B, | major histocompatibility complex, | 25-32 | 24\|25 | B51 | 0.197335344 |
| | -C, -G | class I, A, B, C or G | | | | |
| NPYDSVKKI | UBD | ubiquitin D | 25-33 | no | B51 | 0.1906812 |
| HLINYIIFL | TMEM41B | transmembrane protein 41B | 240-248 | no | A02 | 0.172463768 |
| YVPRAILV | TUBB3 | tubulin, beta 3 | 59-66 | no | B51 | 0.142930066 |
| TLAEIAKVEL | NONO | non-POU domain containing, | 120-129 | no | A02 | 0.137804878 |
| | | octamer-binding | | | | |
| DALDVANKIGII | RPL23A | ribosomal protein L23a | 145-156 | no | B51 | 0.129497908 |
| RIIEETLAL | ARPC2 | actin related protein 2/3 complex, | 9-17 | no | A02 | 0.121783877 |
| | | subunit 2, 34 kDa | | | | |
| DGLVVLKI | EIF3S3 | eukaryotic translation initiation factor | 42-49 | no | B51 | 0.11915734 |
| | | 3, subunit 3 | | | | |
| MAPRTLLL | HLA-A, -B, | major histocompatibility complex, | 4-11 | 24\|25 | B51 | 0.115029632 |
| | -C | class I, A, B or C | | | | |
| VMAPRTLVL | HLA-A | major histocompatibility complex, | 3-11 | 24\|25 | B51 | 0.106570873 |
| | | class I, A | | | | |
| DVANKIGII | RPL23A | ribosomal protein L23a | 148-156 | no | B51 | n.d. |
| LDVPTAAVQA | IFI30 | interferon, gamma-inducible protein | 17-26 | 26\|27 | A02 | n.d. |
| | | 30 | | | | |

### Methods and Materials

### Cell Lines and Mice

Most cell lines used in this study were previously described (16): RMA is a MuLV-induced lymphoma, RMA-S is a TAP2-deficient counterpart of RMA (45), FBL-3 is an erythroleukemia, EL-4 is a dimethylbenzanthracene-induced thymoma, 786 and 771 are MCF1233 MuLV-induced B-cell lymphomas, B16 is a melanoma, TC1 cells are HPV16 E6 and E7 expressing fibroblasts, MC38 and CMT93 are chemically-induced colon carcinomas. C4.4-25⁻ is a β2m deficient variant of EL4 (46). RMA-S.B7-1 is a CD80 transfectant of RNA-S (13). EC7.1 is a K^{b}- and D^{b}-negative variant of RMA-S 21. TAP1^{-/-} and wild type mouse embryo fibroblasts were immortalized by the adenovirus type 5 E1 gene (clone XC3). TAP1^{-/-} and TAP1^{-/-}β2m^{-/-} fibrosarcomas were induced with methylcholantrene (MCB6TAP line). Lipopolysaccharide (LPS) blasts were obtained by culturing spleen cells for three days in the presence of 10 μg/ml LPS (E. Coli 0111:B4, Difco Laboratories, Detroit). All cell lines are from C57BL/6 (B6, H-2b) mice and were cultured in Iscove's modified Dulbecco's medium (Biowhittaker Europe, Verviers, Belgium), supplemented with 8% heat-inactivated fetal calf serum (Gibco BRL, Breda, the Netherlands), 2 mM L-glutamine (ICN Biomedicals Inc., Costa Mesa, Calif.), 100 IU/ml penicillin (Yamanouchi Pharma, Leiderdorp, the Netherlands), and 30 μM 2-mercapto-ethanol (Merck, Darmstadt, Germany) at 37° C. in humidified air with 5% CO2.

C57BL/6 (B6) mice were bred and obtained from TNO-PG breeding facility (Leiden, The Netherlands). TAP1^{-/-} mice (9) were purchased from Jackson Laboratories, Tapasin-knockout mice were kindly provided by Dr. G. J. Hämmerling (20). All mice were backcrossed to B6 and kept under SPF conditions in the animal facility of the Leiden University Medical Center or the Microbiology and Tumor Biology Center in Stockholm.

### Generation of Long-Term CTL Clones, Adoptive Transfer and Peptide Immunization

CTL cultures directed against RMA-S were derived from spleen cells of B6 mice immunized three times subcutaneously (s.c.) with 10⁷ irradiated RMA-S.B7 tumor cells. Generation of CTL lines and clones is previously described (47). The control CTL clones are specific for RMA and recognize the TAP-dependent viral peptide CCLCLTVFL (MuLV^{gag}) or tumor peptide NKGENAQAI (16).

For adoptive transfer experiments, B6 mice were injected s.c. with 5×10⁵ RMA-S tumor cells in 200 μl of saline solution. On the same day 20×10⁶ CTL in 200 μl saline were injected intravenously (i.v.) with 10⁵ Cetus Units of recombinant human IL-2 in a s.c. depot, emulsified in Incomplete Freund's Adjuvants (IFA). An additional injection of IL-2 was given one week after CTL transfer.

B6 mice were immunized with synthetic peptides by s.c. injections of 100 μg of peptide, 50 μg T-helper peptide EPLTSLTPRCNTAWNRLKL 29 and 40 μg Toll-Like Receptor 9 ligand CpG oligonucleotides 28 per mouse in a total volume of 200 μl PBS. Mice were challenged s.c. at the opposite flank with 8×10⁵ RMA-S cells in PBS.

### CTL Assays

Cytolytic activity of CTL was measured by chromium (⁵¹Cr) release assay, as described before (16). CTL reactivity was also measured by IFNγ release. CTL (5×10³) were cocultured with different amounts of stimulator cells (indicated in each figure) in the presence of 5 Cetus Units per ml recombinant IL-2 for 18 to 24 hours, unless otherwise indicated. IFNγ content of supernatants was measured by sandwich ELISA technique (at OD 415) as described (47). For antibody blocking, CTL were pre-treated with 20 μg/ml antibodies, washed and added as responders to target cells for IFNγ release. The following antibodies were used: anti-CD3 (Fab2 fragments of 145-2C11), anti-CD4 (GK1.4) and anti-CD8 (2.43).

### Flowcytometry Analysis

Cells were stained using directly labeled monoclonal antibodies according to standard procedures. The following antibodies were purchased from PharMingen: anti-CD3 (145-2C11), anti-CD4 (GK1.5), anti-CD8α (Ly2), anti-CD8β (Ly-3.2), anti-Vα2 (B20.1), anti-Vα3.2 (RR5.16), anti-TCR Vβ kit, anti-NK1.1 (PK136), DX5, anti-Thy1 (G7), anti-CD16 (2.4G2), anti-Ly49A (A1), anti-Ly49C/I (5E6), anti-Ly49G2 (LGL-1), anti-Ly49D (4E5). Flow cytometry analysis was performed using a FACS Vantage (Becton Dickinson) and analysed using CellQuest software.

Treatment with Proteasome Inhibitors, IFNγ or Antisense Oligonucleotides

To block the proteolytic activity of the proteasome, 1-2×10⁶ targets cells were incubated with 20 μM lactacystin, 20 μM NLVS (4-hydroxy-5-iodo-3-nitrophenylacetyl-leu-leu-leu-vinylsulfone; NIP-L₃VS) or 100 μM LLnL (N-acetyl-L-leucyl-L-leucyl-leucyl-L-norleucinal) (Calbiochem, Breda, The Netherlands) in 2 ml of complete culture medium at 37° C. for 2 hours. Cells were washed with saline buffer and acid stripped for 2 min using cold citrate buffer (pH=3.0). Thereafter, cells were washed again, incubated for a second period of 2 hours with proteasome inhibitors, labeled with chromium and used as targets in a cytolytic assay. Control targets were only briefly acid stripped.

RMA and 786 tumor cells were treated with 100 IU/ml IFN gamma for 48 h in 24-well plates. Cells were harvested, washed three times and used as targets for CTL.

MC38 and B16 tumor cells were treated with ‘morpholino’ oligonucleotides (48) (Gene Tools, OR, USA) encoding the antisense sequence of mouse TAP1 (5′-AGAGTCTGGTCCTAGCCTGGGA-3′) or control sequence (5′-GGCGAGAAGCTCAGCCATTTAGGG-3′). Oligonucleotides were loaded into target cells by osmotic shock as recommended by the manufacturer, and assayed after nine days.

### RT-PCR

Total RNA from 10⁷ T cells was isolated using TRIzol™ according to the manufacturer's recommendation (Gibco BRL). cDNA was generated by oligoT-primed RNA using AMV-reverse transcriptase (Promega). Reaction was heat-inactivated, diluted in water and stored at −20° C. until use. For Ly49 RT-PCR the following generic primers were used 5′-CAATGGCCCATCTAAACTTG-3′ and 5′-CCAGTTTCTTCCCACAAATACA-3′, generating a product of 149 bp. This PCR reaction detects most Ly49 family members. For CD94 cDNA the following primers were used 5′-ATGGCAGTTTCTAGGATCACTCGG-3′ and 5′-GCTGGAATTCTGCGAAGCACAGA-3′, resulting in a product of 280 bp. The PCR primers for the NKG2 genes have been published (49). The applied PCR primers for cloning Trh4 long transcript were 5′-ATGGCGACTGCAGCAGCAGCGGAAACCC-3′ and 5′-CTACATCACTGCGGTCATCCTTAGACACATGCAAAGG-3′. PCR products were directly cloned with the TOPO TA Cloning® kit (Invitrogen) and sequenced using standard procedures. Detection of Trh4 short and long transcripts was performed by rt-PCR using a shared upstream primer 5′-GCAGACCCCTTACTGGAAGCTGCC-3′ and specific downstream primers 5′-CGGTCATCCTTAGACACATGCAAAGG-3′ (long) or 5′-CTGCGGTCATCCTTAGACACCTTTCC-3′ (short).

### Synthetic Peptide Library

A synthetic peptide library was synthesized on hybrid beads. Hybrid beads were generated by coupling a mixture of Fmoc-Nle-OH and the 3-(4-hydroxymethyl-phenoxy)propionic acid ester of Fmoc-Val-OH to a TentagelS amine resin (loading 0.26 mmol/g, particle size 130 μm). A peptide library containing about 650,000 peptides of the general structure XLXXXXXXV (X is random position) was synthesized according to a mix and split one-bead-one-peptide protocol. The peptide library, of which each bead contained about 25% acid-stable attached peptide (ASA-P) and 75% acid-labile attached peptide (ALA-P), was suspended in a mixture of dichloroethane/acetonitrile 82/18 and divided into pools (about 2,200 peptides/well) in 3 96 wells polypropylene filtration plates. Beads were washed with acetonitrile and ether, respectively, and air-dried. Each peptide pool was treated for 30 minutes with 150 μl of a mixture of 15% trifluoroacetic acid, 40% trifluoroethanol, 40% acetonitrile and 5% water and the liquid was isolated by filtration. The beads were washed with 75 μl of the same mixture and the combined filtrates were dried in a stream of nitrogen. This procedure cleaves about ⅓ of the ALA-P. The beads were neutralized by several washings with 0.25 M Tris in water/acetonitril 1:1, pH=8), water, acetonitril and ether, respectively. Side-chain deprotection of the peptides in the dried filtrate was performed by a 2 hr incubation with 150 μl of a mixture of 90% trifluoroacetic acid, 5% water and 5% ethanethiol and subsequent drying in a stream of nitrogen. Each peptide pool was dissolved in 5 μl dimethylsulfoxide after which 100 μl of a 30 mM solution of sodiumphosphate in water (pH=7.5) was added. Dissolved peptide pools were stored at −20° C. until T cell screening. Each peptide pool was screened in a TEIPP-specific CTL assay using T2.D^{b} or T2.K^{b} target cells. Pools of beads that corresponded to peptide pools with T cell stimulating activity were divided in one 96 wells polypropylene filtration plate (about 23 peptides/well). Cleavage and work-up of the second ⅓ of the ALA-P was performed using the same procedure as described above. Again beads that corresponded to peptide pools with TEIPP-specific CTL were divided in a 96 well polypropylene filtration plate in limiting dilution (0 of 1 beads per well). The remaining of the ALA-P was cleaved from the beads by a 2 hr incubation with 150 μl of a mixture of 90% trifluoroacetic acid, 5% water and 5% ethanethiol and subsequent drying in a stream of nitrogen. Individual beads that correspond to TEIPP-specific CTL activity of the related peptide were sequenced by Edman degradation in order to obtain the amino acid sequence of the T cell stimulating mimotope. The whole screening process was validated by resynthesizing the mimotopes and subsequent testing.

### Peptide Elution

Peptides were eluted out of purified H-2D^{b} or H-2K^{b} molecules as previously described (16) with minor modifications. Immunoprecipitation was performed with protein A beads covalently coupled with anti-D^{b} mAb 28-14-8S or anti-K^{b} mAb B8-24-3 from lysates of 10¹¹ RMA-S cells. Eluted peptides were fractionated using reverse phase micro C₂C₁₈ HPLC (Smart system Amersham). Buffer A was 0.1% trifluoroacetic acid in water; buffer B was 0.1% trifluoroacetic acid in acetonitrile. Alternatively, peptides were acid extracted from purified HLA-A*02 and HLA-B*51 molecules of 10¹⁰ human B-cells (lines LCL721.174 (TAP-negative) and LCL721.45 (TAP-positive)). Two pools of peptides were subjected to our recently developed guanidination and nicotinylation method of isotype labelling (Lemmel et al, Nature Biotechnology 22:450, 2004). Equal amounts of both peptide pools were mixed and subsequently run on HPLC for reduction of peptide complexity. Mass spectrometry analysis using ESI-MS was applied to determine differential presented peptides and MS/MS spectra from peptides of interest were recorded in order to obtain their amino acid sequence.

### Mass Spectrometry

Electrospray ionization mass spectrometry was performed on a hybrid quadrupole time-of-flight mass spectrometer (Q-TOF1 and Q-TOF Ultima, Micromass) equipped with an on-line nanoelectrospray source, as described before (16, 50). Tandem MS of the Trh4 derived peptide was performed in a Bruker HCT^{plus} Ion-trap. The ion 528.3 m/z was selected with a window of 4 Da and sequenced by MS/MS.

### REFERENCES

1. Harty, J. T., Tvinnereim, A. R. & White, D. W. CD8⁺ T cell effector mechanisms in resistance to infection. Annu Rev Immunol 18, 275-308 (2000).
2. Melief, C. J. M. et al. Strategies for immunotherapy of cancer. Adv. Immunol. 75, 235-282 (2000).
3. Vossen, M. T., Westerhout, E. M., Soderberg-Naucler, C. & Wiertz, E. J. Viral immune evasion: a masterpiece of evolution. Immunogenetics 54, 527-42 (2002).
4. Khong, H. T. & Restifo, N. P. Natural selection of tumor variants in the generation of “tumor escape” phenotypes. Nat Immunol 3, 999-1005 (2002).
5. Hicklin, D. J., Marincola, F. M. & Ferrone, S. HLA class I antigen downregulation in human cancers: T-cell immunotherapy revives an old story. Mol. Med. Today 5, 178-186 (1999).
6. Seliger, B., Maeurer, M. J. & Ferrone, S. TAP off-Tumors on. Immunology Today 18, 292 (1997).
7. Marincola, F. M., Jaffee, E. M., Hicklin, D. J. & Ferrone, S. Escape of human solid tumors from T-cell recognition: molecular mechanisms and functional significance. Adv. Immunology 74, 181-273 (2000).
8. Cresswell, P., Bangia, N., Dick, T. & Diedrich, G. The nature of the MHC class I peptide loading complex. Immunol Rev 172, 21-8 (1999).
9. Van Kaer, L., Ashton-Rickardt, P. G., Ploegh, H. L. & Tonegawa, S. TAP1 mutant mice are deficient in antigen presentation, surface class I molecules, and CD4⁻8⁺ T cells. Cell 71, 1205-1214 (1992).
10. Chen, H. L. et al. A functionally defective allele of TAP1 results in loss of MHC class I antigen presentation in a human lung cancer. Nature Genetics 13, 210-213 (1996).
11. Evans, L. K. et al. Antigen processing defects in cervical carcinomas limit the presentation of a CTL epitope from human papillomavirus 16 E6. J. Immunol. 167, 5420-5428 (2001).
12. Alimonti, J. et al. TAP expression provides a general method for improving the recognition of malignant cells in vivo. Nature Biotechnology 18, 515-520 (2000).
13. Wolpert, E. Z. et al. Generation of CD8⁺ T cells specific for transporter associated with antigen processing deficient cells. Proc. Natl. Acad. Sci. USA 94, 11496-11501 (1997).
14. Vivier, E. & Anfossi, N. Inhibitory NK-cell receptors on T cells: witness of the past, actors of the future. Nat. Rev. Immunol. 4, 190-198 (2004).
15. Sijts, E. J. A. M., Ossendorp, F., Mengedé, E. A. M., Van den Elsen, P. J. & Melief, C. J. M. An immunodominant MCF murine leukemia virus-encoded CTL epitope, identified by its MHC class I-binding motif, explains MuLV type specificity of MCF-directed CTL. J. Immunol. 152, 106-116 (1994).
16. Van Hall, T. et al. Identification of a novel tumor-specific CTL epitope presented by RMA, EL-4, and MBL-2 lymphomas reveals their common origin. J. Immunol. 165, 869-877 (2000).
17. Farrar, M. A. & Schreiber, R. D. The molecular cell biology of interferon-γ and its receptor. Ann. Rev. Immunol. 11, 571-611 (1993).
18. Attaya, M. et al. Ham-2 corrects the class I antigen-processing defect in RMA-S cells. Nature 355, 647-649 (1992).
19. Reits, E. A., Vos, J. C., Gromme, M. & Neefjes, J. The major substrates for TAP in vivo are derived from newly synthesized proteins. Nature 404, 774-778 (2000).
20. Garbi, N. et al. Impaired immune responses and altered peptide repertoire in tapasin-deficient mice. Nature Immunol. 1, 234-238 (2000).
21. Howell, D. et al. Heterogeneity of RMA-S cell line: derivatives of RMA-S cells lacking H2-K^{b} and H2-D^{b} expression. Immunogenetics 52, 150-154 (2000).
22. Chiang, E. Y., Henson, M. & Stroynowski, I. Correction of defects responsible for impaired Qa-2 class Ib MHC expression on melanoma cells protects mice from tumor growth. J. Immunol. 170, 4515-4523 (2003).
23. Wei, M. L. & Cresswell, P. HLA-A2 molecules in an antigen-processing mutant cell contain signal sequence-derived peptides. Nature 356, 443-446 (1992).
24. Henderson, R. A. et al. HLA-A2.1-associated peptides from a mutant cell line: a second pathway of antigen presentation. Science 255, 1264-1266 (1992).
25. Winter, E. & Ponting, C. P. TRAM, LAG1 and CLN8: members of a novel family of lipid-sensing domains? Trends Biochem Sci 27, 381-3 (2002).
26. Riebeling, C., Allegood, J. C., Wang, E., Merrill, A. H., Jr. & Futerman, A. H. Two mammalian longevity assurance gene (LAG1) family members, trh1 and trh4, regulate dihydroceramide synthesis using different fatty acyl-CoA donors. J Biol Chem 278, 43452-9 (2003).
27. Snyder, H. L., Bacik, I., Yewdell, J. W., Behrens, T. W. & Bennink, J. R. Promiscuous liberation of MHC-class I-binding peptides from the C termini of membrane and soluble proteins in the secretory pathway. Eur J Immunol 28, 1339-46 (1998).
28. Zwaveling, S. et al. Established human papillomavirus type 16-expressing tumors are effectively eradicated following vaccination with long peptides. J. Immunol. 169, 350-358 (2002).
29. Ossendorp, F., Mengede, E., Camps, M., Filius, R. & Melief, C. J. M. Specific T-helper cell requirement for optimal induction of cytotoxic T-lymphocytes against major histocompatibility complex class-II negative tumors. J Exp Med 187, 693-702 (1998).
30. De Silva, A. D. et al. Thermolabile H-2K^{b} molecules expressed by transporter associated with antigen processing-deficient RMA-S cells are occupied by low-affinity peptides. J. Immunol. 163, 4413-4420 (1999).
31. Yewdell, J. W., Reits, E. & Neefjes, J. J. Making sense of mass destruction: quantitating MHC class I antigen presentation. Nat. Rev. Immunol. 3, 952-961 (2003).
32. Wu, T. C., Huang, A. Y., Jaffee, E. M., Levitsky, H. I. & Pardoll, D. M. A reassessment of the role of B7-1 expression in tumor rejection. J. Exp. Med. 182, 1415-1421 (1995).
33. Snyder, H. L. et al. Two novel routes of transporter associated with antigen processing (TAP)-independent major histocompatibility complex class I antigen processing. J Exp Med 186, 1087-98 (1997).
34. Aldrich, C. J. et al. Positive selection of self- and alloreactive CD8⁺ T cells in TAP-1 mutant mice. Proc. Natl. Acad. Sci. USA 91, 6525-6528 (1994).
35. Sandberg, J. K., Chambers, B. J., Van Kaer, L., Karre, K. & Ljunggren, H. G. TAP1-deficient mice select a CD8⁺ T cell repertoire that displays both diversity and peptide specificity. Eur. J. Immunol. 26, 288-293 (1996).
36. Jensen, P. E., Sullivan, B. A., Reed-Loisel, L. M. & Weber, D. A. Qa-1, a nonclassical class I histocompatibility molecule with roles in innate and adaptive immunity. Immunol. Res. 29, 81-92 (2004).
37. Tallquist, M. D., Weaver, A. J. & Pease, L. R. Degenerate recognition of alloantigenic peptides on a positive-selecting class I molecule. J Immunol 160, 802-9 (1998).
38. Heemskerk, M. H. et al. Dual HLA class I and class II restricted recognition of alloreactive T lymphocytes mediated by a single T cell receptor complex. Proc. Natl. Acad. Sci. USA 98, 6806-6811 (2001).
39. Basu, D., Horvath, S., O'Mara, L., Donermeyer, D. & Allen, P. M. Two MHC surface amino acid differences distinguish foreign peptide recognition from autoantigen specificity. J. Immunol. 166, 4005-4011 (2001).
40. Wilson, D. B. et al. Specificity and degeneracy of T cells. Mol Immunol 40, 1047-55 (2004).
41. Holler, P. D. & Kranz, D. M. T cell receptors: affinities, cross-reactivities, and a conformer model. Mol Immunol 40, 1027-31 (2004).
42. Johnsen, A. K., Templeton, D. J., Sy, M. S. & Harding, C. V. Deficiency of transporter for antigen presentation (TAP) in tumor cells allows evasion of immune surveillance and increased tumorigenesis. J. Immunol. 163, 4224-4231 (1999).
43. Qin, Z. et al. Increased tumorigenicity, but unchanged immunogenicity, of transporter for antigen presentation 1-deficient tumors. Cancer Res. 62, 2856-2860 (2002).
44. Georgopoulos, N. T., Proffitt, J. L. & Blair, G. E. Transcriptional regulation of the major histocompatibility complex (MHC) class I heavy chain, TAP1 and LMP2 genes by the human papillomavirus (HPV) type 6b, and 18 E7 oncoproteins. Oncogene 19, 4930-4935 (2000).
45. Kaire, K., Ljunggren, H. G., Piontek, G. & Kiessling, R. Selective rejection of H-2-deficient lymphoma variants suggests alternative immune defense strategy. Nature 319, 675-678 (1986).
46. Glas, R., Sturmhofel, K., Hammerling, G. J., Karre, K. & Ljunggren, H. G. Restoration of a tumorigenic phenotype by beta 2-microglobulin transfection to EL-4 mutant cells. J. Exp. Med. 175, 843-846 (1992).
47. Van Hall, T. et al. Differential influence on cytotoxic T-lymphocyte epitope presentation by controlled expression of either proteasome immunosubunits or PA28. J. Exp. Med. 192, 483-494 (2000).
48. Summerton, J. & Weller, D. Morpholino antisense oligomers: design, preparation, and properties. Antisense & nucl. ac. drug dev. 7, 187-195 (1997).
49. Toomey, J. A. et al. Stochastic acquisition of Qa1 receptors during the development of fetal NK cells in vitro accounts in part but not in whole for the ability of these cells to distinguish between class I-sufficient and class I-deficient targets. J. Immunol. 163, 3176-3184 (1999).
50. Meiring, H. D., Van der Heeft, E., Ten Hove, G. J. & De Jong, A. P. Nanoscale LC-MS: technical design and applications to peptide and protein analysis. J. Sep. Sci. 25, 557 (2002).
51. Van Hall, T. et al. Cryptic open reading frames in plasmid vector backbone sequences can provide highly immunogenic cytotoxic T-lymphocyte epitopes. Canc. Res. 58, 3087-3093 (1998).

1-18. (canceled)

19. A method for identifying an MHC class I binding peptide comprising a TEIPP epitope, comprising:
a) obtaining a cell deficient in TAP transport; AND
b1) inducing and isolating a TEIPP specific T-cell against the TAP deficient cells in a mammal;
b2) screening a peptide library with the isolated TEIPP specific T-cell and identifying one or more mimotope sequences that are recognized by the T-cell;
b3) determining the T-cell-specific recognition motif by substituting single amino acids in the one or more mimotope sequences identified in (c); and
b4) screening a database of naturally occurring polypeptide sequences with the recognition motif to identify a naturally occurring peptide that matches the motif; OR,
c) performing mass-spectrometric analysis of purified natural peptides displayed on MHC molecules obtained from TAP deficient cells.

20. The method according to claim 19 wherein (b2) comprises screening of peptides physically linked to a solid carrier with acid-labile and/or acid-stable chemical linkage.

21. The method according to claim 19 wherein step (c) comprises performing differential analysis of TAP proficient and TAP deficient cells derived from a single host organism.

22. The method according to claim 21 wherein the analysis comprises differential mass spectrometric analysis using isotope labeling of TAP deficient and TAP proficient cells.

23. A peptide comprising an epitope obtained from the method according to any of claim 19, wherein the peptide is:
a) optionally trimmed or extended to comprise between 14 and 120 amino acids in length, and/or
b) optionally flanked with process-dependent sequences.

24. The peptide according to claim 23 in the context of an MHC class I molecule.

25. The peptide according to claim 24 wherein the MHC class I molecule is a non-classical MHC class I molecule, preferably a HLA-E molecule.

26. The peptide according to claim 25 wherein the non-classical MHC class I molecule is a HLA-E molecule.

27. The peptide according to claim 23 wherein the peptide further comprises a T helper epitope that is presentable by MHC class II molecules.

28. The peptide according to claim 23 comprising a sequence selected from the group consisting of SEQ ID Nos. 1 to 35.

29. A nucleic acid sequence encoding a peptide comprising an epitope obtained from the method according to any of claim 19, wherein the peptide is:
a) optionally trimmed or extended to comprise between 14 and 120 amino acids in length, and/or
b) optionally flanked with process-dependent sequences.

30. The nucleic acid sequence according to claim 28 comprising a nucleic acid vector capable of conferring expression of the epitope in a host cell.

31. A host cell comprising the nucleic acid comprising the nucleic acid sequence as defined in claim 28.

32. A host cell comprising the nucleic acid comprising the vector as defined in claim 29.

33. A composition for eliciting an immune response in a subject comprising at least a peptide comprising an epitope according to claim 23, and optionally one or more peptides comprising a tumor or virus specific epitope and/or an adjuvant.

34. The composition according to claim 33, wherein the immune response against TAP impaired or deficient cells

35. The composition according to claim 34 wherein the TAP impaired or deficient cell is a tumor cell or a virally infected cell.

36. The composition according to claim 33, wherein the peptide comprising the epitope is expressed from a nucleic acid sequence or a cell capable of expressing the peptide.

37. The composition according to claim 33, wherein the peptide is a signal peptides derived from or cleaved off upon transport of a protein over a membrane.

38. A method of eliciting an immune response in a subject comprising administering to the subject a peptide comprising an epitope according to claim 23, and optionally one or more peptides comprising a tumor or virus specific epitope and/or an adjuvant.
